(19) United States
(12) Patent Application Publication
(10) Pub. No.: US 2023/0310314 A1
(43) Pub. Date: Oct. 5, 2023

(54) MUCOADHESIVE COMPOSITIONS FOR DIRECTIONAL DELIVERY OF ACTIVE AGENTS

(71) Applicant: AltrixBio, Inc., Cambridge, MA (US)

(72) Inventors: Yuhan LEE, Cambridge, MA (US); Shardool JAIN, Cambridge, MA (US); Jeffrey KARP, Cambridge, MA (US); Ali TAVAKKOLI, Cambridge, MA (US)

(73) Assignee: AltrixBio, Inc., Cambridge, MA (US)

(21) Appl. No.: 18/175,442

(22) Filed: Feb. 27, 2023

Related U.S. Application Data
(60) Provisional application No. 63/314,575, filed on Feb. 28, 2022.

(51) Int. Cl. A61K 9/00; A61K 47/26; A61K 38/26; A61K 38/28; A61K 31/155; A61K 47/32

(52) U.S. Cl. CPC A61K 9/0065; A61K 31/155; A61K 38/26; A61K 38/28; A61K 47/26; A61K 47/32

(57) ABSTRACT

Compositions formulated for release within the gastrointestinal tract and related methods are discussed. The composition may include a mucoadhesive agent and an active agent. The composition may be formulated for release of the active agent in a first direction while inhibiting release of the active agent in a second direction opposite the first direction.

### CROSS-REFERENCE TO RELATED APPLICATIONS

This application claims priority to U.S. Provisional Application No. 63/314,575, filed on Feb. 28, 2022, which is incorporated by reference herein in its entirety.

### TECHNICAL FIELD

The present disclosure relates generally to therapeutic compositions, including compositions formulated for release within the gastrointestinal (GI) tract.

### BACKGROUND

The administration of drugs (e.g., active agents such as pharmaceuticals and biologics) via the gastrointestinal tract presents challenges with respect to drug bioavailability and efficacy. For example, while traveling through the gastrointestinal (GI) tract, drugs may be prematurely or belatedly released. In addition, low pH and enzymatic degradation of the drugs within the GI tract can significantly impact bioavailability of drugs.

### SUMMARY

The present disclosure includes compositions formulated for release within the GI tract, e.g., for targeted delivery of an active agent to the GI tract. For example, the present disclosure includes a composition comprising at least one mucoadhesive agent and at least one active agent, wherein the composition is formulated for release of the active agent in a first direction while inhibiting release of the active agent(s) in a second direction opposite the first direct, and wherein the composition is formulated for administration to a subject for release in a gastrointestinal tract of the subject. The mucoadhesive agent may comprise an aluminum cross-linked sulfated agent that forms a paste with an aqueous liquid in the absence of acid. In some examples herein, the mucoadhesive agent and the active agent may be present in discrete layers or regions of the composition. Optionally, the mucoadhesive agent and the active agent may be arranged as layers in an axial or radial configuration. According to some aspects, the composition may be formulated as an osmotic pump, a drug delivery device, a tablet, a plurality of tablets, a capsule, or a plurality of capsules for oral administration.

In some examples, the composition includes an enteric polymer, an anti-adhesive agent, or both. In at least one example, the composition is in the form of a tablet that includes a first layer comprising the mucoadhesive agent a second layer comprising the active agent, optionally wherein the first and second layers have an axial or radial configuration. The composition may further comprise a spacer material between the mucoadhesive agent and the active agent, optionally wherein the spacer material comprises a polymer, copolymer, binder, permeation enhancer, disintegrant, surfactant, or combination thereof. For example, the composition may be in the form of a tablet that includes a first layer comprising the mucoadhesive agent, a second later comprising the spacer material, and a third layer comprising the active agent. The active agent may be mixed with a polymer, copolymer, permeation enhancer, surfactant, or combination thereof. In some examples, the spacer material comprises a cellulose polymer such as hydroxypropylcellulose or microcrystalline cellulose, and/or polyvinylpyrrodlidone.

According to some aspects, the mucoadhesive agent is a first mucoadhesive agent and the composition further comprises a second mucoadhesive agent different from the first mucoadhesive agent. The mucoadhesive agent may comprise aluminum hydroxide crosslinked with sulfated sucrose, sulfated dextran, sulfated dextrin, sulfated amylopectin, sulfated amylose, sulfated cellulose, carrageenan, chondroitin sulfate, glucose sulfate, sucrose sulfate, heparin, heparin sulfate, or raffinose sulfate. The active agent may comprise a protein, peptide, carbohydrate, lipid, nutraceutical, small molecule, vitamin, steroid, enzyme, nucleotide, receptor, neurotransmitter, hormone, cytokine, cell adhesion sequence, extracellular matrix component, growth factor, chemotactic factor, antibody, vaccine, hapten, interferon, ribozyme, antisense agent, plasmid, aptamer, bacteria, virus, or a combination thereof. In some examples, the active agent may comprise an anti-inflammatory agent, analgesic, antimicrobial agent, steroid, stem cell activating molecule, or regenerative therapeutic agent. In at least one example, the active agent comprises a peptide such as glucagon-like peptide 1 (GLP-1), insulin, or metformin.

Also disclosed herein is a method of treating a subject, comprising administering the composition as described above or elsewhere herein to the subject. In some examples, the subject has a health condition chosen from diabetes, obesity, Crohn’s disease, nonalcoholic steatohepatitis, nonalcoholic fatty liver disease, Barrett’s esophagus, or cancer, and the active agent treats the health condition. The composition may form a mucoadhesive coating on a stomach, esophagus, small intestine, or large intestine of the subject. For example, the mucoadhesive coating may form on healthy tissue or tissue that is diseased, such as ulcerated tissue. In some examples, the composition may comprise a plurality of tablets or a plurality of capsules. Additionally or alternatively, the composition may comprise a plurality of tablets within a capsule. In some examples, the composition may comprise an enteric layer or coating.

The present application further includes a composition comprising a mucoadhesive agent and an active agent; wherein the composition is formulated for release of the active agent in a first direction while inhibiting release of the active agent in a second direction opposite the first direction; wherein the composition is formulated for administration to a subject for release in a gastrointestinal tract of the subject; and wherein the composition contains an agent that hydrates to form a transient mucoadhesive paste at any pH. The viscosity of the paste may range from about 0.5-5 Pa.s at about 25° C., or 0.1-50 Pa.s or higher at about 25° C.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 illustrates an exemplary composition attached to mucosal tissue, according to some aspects of the present disclosure.

FIG. 2 illustrates an exemplary composition with multiple layers arranged axially, according to some aspects of the present disclosure.

FIG. 3 illustrates another exemplary composition with multiple layers arranged axially, according to some aspects of the present disclosure.

FIG. 4 illustrates another exemplary composition with multiple layers arranged axially, according to some aspects of the present disclosure.

FIG. 5 illustrates an exemplary composition with multiple layers forming a paste, according to some aspects of the present disclosure.

FIG. 6 illustrates an exemplary composition with multiple layers arranged radially, according to some aspects of the present disclosure.

FIG. 7 illustrate an exemplary composition with multiple layers arranged axially and different scenarios for the composition contacting mucosal tissue, according to some aspects of the present disclosure.

FIG. 8 illustrates an exemplary composition comprising a plurality of tablets with multiple layers arranged axially, according to some aspects of the present disclosure.

FIG. 9 illustrates an exemplary composition comprising a plurality of tablets with multiple layers arranged radially, according to some aspects of the present disclosure.

FIG. 10 illustrates another exemplary composition comprising a plurality of tablets with multiple layers arranged radially, according to some aspects of the present disclosure.

FIG. 11 illustrates another exemplary composition comprising a plurality of tablets with multiple layers arranged radially, according to some aspects of the present disclosure.

FIG. 12 illustrates an exemplary composition comprising a plurality of tablets with multiple layers arranged radially, according to some aspects of the present disclosure.

FIG. 13 shows results from a study on release of a composition comprising a mucoadhesive agent as compared to a control, discussed in Example 1.

FIG. 14 shows results from a study on release of a composition comprising a mucoadhesive agent as compared to a control, discussed in Example 2.

FIG. 15 shows results from a study on release of a composition comprising a mucoadhesive agent as compared to a control, discussed in Example 3.

FIG. 16 shows results from a study on release of a composition comprising a mucoadhesive agent as compared to a control, discussed in Example 4.

FIG. 17 shows results from a study on release of a composition comprising a mucoadhesive agent as compared to a control, discussed in Example 5.

FIG. 18 shows results from a study on release of test compositions comprising a mucoadhesive agent as compared to a control, discussed in Example 6.

FIG. 19 shows results from a study on release of test compositions comprising a mucoadhesive agent as compared to a control, discussed in Example 7.

FIG. 20 shows results from a study on release of a composition comprising a mucoadhesive agent as compared to a control, discussed in Example 8.

FIG. 21 shows results from a study on release of a composition comprising a mucoadhesive agent as compared to a control, discussed in Example 9.

FIG. 22 shows results from a study on release of test compositions comprising a mucoadhesive agent under static and dynamic conditions, discussed in Example 10.

FIG. 23 shows results from a study on release of a composition comprising a mucoadhesive agent as compared to a control, discussed in Example 11.

FIG. 24 shows results from a study on release of a composition comprising a mucoadhesive agent as compared to a control, discussed in Example 12.

FIG. 25 shows results from a study on release of compositions comprising a mucoadhesive agent as compared to a control, discussed in Example 13.

### DETAILED DESCRIPTION

Particular aspects of the present disclosure are described in greater detail below. The terms and definitions provided herein control, if in conflict with terms and/or definitions incorporated by reference.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise. The terms “approximately” and “about” refer to being nearly the same as a referenced number or value. As used herein, the terms “approximately” and “about” should be understood to encompass ± 5% of a specified amount or value.

The present disclosure includes compositions formulated for release within the GI tract, e.g., for targeted delivery of an active agent to the GI tract. This targeted delivery may include a one-directional delivery, such that the active agent may be released in the direction towards adjacent or proximate tissue of the GI tract and prevented or otherwise inhibited from release in the opposite direction.

Compositions described herein may provide a suitable matrix for encapsulation and protection of drugs from harsh GI environments. The compositions herein may comprise at least one mucoadhesive component (also referred to herein as a mucoadhesive agent) and an active agent. For example, the mucoadhesive component(s) may comprise a sulfated agent as disclosed in U.S. Pat. No. 10,973,846, incorporated by reference herein. In at least one example, the mucoadhesive agent comprises acid pretreated sucralfate as disclosed in U.S. Pat. No. 10,973,846. The composition may be formulated such that the mucoadhesive component and the active agent are present in discrete layers or regions of the composition, e.g., rather than being mixed together such as in a distributed matrix, wherein the discrete layers or regions provide for one-directional release. In some examples, the composition comprises two or more mucoadhesive agents, which may be different. For example, the composition may comprise a first mucoadhesive agent and a second mucoadhesive agent different from the first mucoadhesive agent.

The compositions herein may provide for one-directional release of the active agent, wherein the mucoadhesive component (e.g., sucralfate) prevents or otherwise inhibits release of the active agent into the GI tract lumen. The mucoadhesive component may serve as a coating or barrier to prevent or otherwise inhibit outward diffusion of the active agent into the GI lumen, allowing the active agent to release towards the GI mucosal tissue surface. Therefore, the active agent may be absorbed through the tissue-composition interface, which may provide for increased absorption. Optionally, the composition may comprise a material or mixture of materials inhibiting water/moisture uptake. For example, the composition may comprise a layer (e.g., an outermost layer or coating) on at least a portion of the composition that inhibits water uptake by the mucoadhesive component and/or the active agent, e.g., preventing premature hydration of the mucoadhesive component. The composition may additionally or alternatively comprise a spacer between the mucoadhesive component and the active agent, as discussed further below. In some examples, the composition may comprise an enteric coating.

The compositions herein may comprise multiple layers of different materials or agents to provide for the desired release of active agent at a target area of tissue. For example, the composition may be a multilayered composition. In some examples, the composition may have an axially layered configuration, e.g., comprising at least a mucoadhesive component in a first layer and an active agent in a second layer. In some examples, the composition comprises a spacer component in a third layer between the first and second layers in an axial direction. In some examples, the layers may be arranged radially, e.g., comprising an active agent as an innermost layer or, core, and a mucoadhesive component forming a layer over the innermost layer, radially outside the innermost layer. The compositions herein may be formulated in various dosage forms, such as tablets, osmotic pumps, drug delivery devices, and capsules, including single unit compositions and multiple unit compositions (e.g., multiple unit compositions such as a plurality of mini-tablets or minicapsules), as discussed further below.

The compositions herein may have one or more of the following benefits: (1) the mucoadhesive agent blocks washout of the active agent(s) to the GI lumen, (2) the composition promotes release of the active agent towards tissue, (3) the composition facilitates absorption of the active agent(s) at the tissue interface, e.g., for improved bioavailability, (4) the composition allows for release at targeted tissue within the GI tract, (5) the composition allows for modulating the release profile (immediate/extended/sustained/delayed) of an active agent in accordance with desired specifications, and/or (6) the composition may allow for release of two or more active agents, e.g., for a target indication.

Further, the compositions herein may include one, two, three, four, or all five of the following characteristics:

1) The composition may be formulated to release most of the active agent within about 1 hour of administration to a subject (e.g., at least 50%, at least 60%, at least 70%, or at least 80%) and/or the composition may be formulated to modulate the contact/residence time to the mucosa of a target area. See examples below wherein release of the active agent may be measured relative to a control and showing top-view time course pictures of morphological changes in the active agent drug depot (e.g., solution or particles etc.).

2) The mucoadhesive agent may form a coating or barrier around the active agent(s) when administered to a subject (e.g., as the composition contacts mucosal tissue). See examples below comparing results due to the presence of a mucoadhesive layer to a control group without the mucoadhesive agent, and top-view time coarse pictures showing the mucoadhesive formation over the active agent drug depot.

3) The mucoadhesive agent does not interfere or has limited interference with the active agent and absorption of the active agent(s) by the tissue. See examples below comparing results due to the presence of a mucoadhesive layer to a control group without the mucoadhesive agent.

4) The composition may have mucoadhesive properties providing for attachment to mucosal tissue; and/or enhance permeation across the mucosal tissue.

5) The active agent(s) do not separate into multiple pieces or particles.

The mucoadhesive component may comprise a sulfated agent, e.g., an aluminum cross-linked sulfated agent. Examples of mucoadhesive agents suitable for the present disclosure include, but are not limited to, aluminum hydroxide crosslinked with sulfated sucrose, sulfated dextran, sulfated dextrin, sulfated amylopectin, sulfated amylose, sulfated cellulose, carrageenan, chondroitin sulfate, glucose sulfate, sucrose sulfate, heparin, heparin sulfate, or raffinose sulfatsucralfate, sulfated dextran, sulfated dextrin, sulfated amylopectin, sulfated amylose, sulfated cellulose, carrageenan, chondroitin sulfate, glucose sulfate, sucrose sulfate, heparin, heparin sulfate, and raffinose sulfate. In some examples, the composition comprises a first mucoadhesive agent comprising aluminum hydroxide crosslinked with sulfated sucrose, sulfated dextran, sulfated dextrin, sulfated amylopectin, sulfated amylose, sulfated cellulose, carrageenan, chondroitin sulfate, glucose sulfate, sucrose sulfate, heparin, heparin sulfate, or raffinose sulfate; and a second mucoadhesive agent different from the first mucoadhesive agent. For example, the second mucoadhesive agent may comprise poly(ethylene glycol), poly(vinyl alcohol), poly(vinyl pirrolidone), poly(acrylic acid), carbopol, poly(hydroethyl methacrylate), chitosan, hydroxyethyl cellulose, hydroxypropyl cellulose, hydroxypropylmethyl cellulose, methylcellulose, or sodium carboxymethyl cellulose. The first and second mucoadhesive agents may be in separate layers of the composition. In at least one example, the first mucoadhesive component comprises aluminum hydroxide crosslinked with sulfated sucrose (e.g., acid-pretreated sucralfate).

In some examples, the mucoadhesive agent comprises viscous material formed upon ex vivo acid pretreatment of sucralfate. Sucralfate is a water-insoluble salt that comprises two oppositely charged polyelectrolytes: anionic sucrose octasulfate and a high molecular weight cationic polyaluminum complex. In the compositions herein, sucralfate may be formulated based on characteristics of tissue of the target area within the GI tract, characteristics of the active agent, and/or the conditions(s) of the subject to be treated. The tissue may be healthy mucosal tissue or mucosal tissue that is afflicted with an ailment, such as an ulcer. In at least one example, the mucoadhesive agent comprises sucralfate capable of forming a mucoadhesive coating on ulcerated tissue. In at least one example, the composition comprises acid pretreated sucralfate capable of forming a mucoadhesive coating on healthy mucosa. For example, the acid-pretreated sucralfate may be formulated so that it can form a paste in the presence of an aqueous solution and in the absence of acid. Acid pretreatment of sucralfate may allow for formation of a mucoadhesive layer on GI tissue, including healthy GI tissue as well as ulcerated GI tissue. The mucoadhesive component may be in paste, gel, or powder form (e.g., the powder being capable of forming a paste in the presence of aqueous liquid), for example. In some examples, the mucoadhesive component may form a paste in situ. For example, the mucoadhesive component may become a transient paste upon contact with an aqueous liquid at any pH.

Some examples may include an optional (e.g., additional) layer comprising a different mucoadhesive component than those described above. Examples of the mucoadhesive agents suitable for this different mucoadhesive component include, but are not limited to, poly(ethylene glycol), poly(vinyl alcohol), poly(vinyl pirrolidone), poly(acrylic acid), carbopol, poly(hydroethyl methacrylate), chitosan, hydroxyethyl cellulose, hydroxypropyl cellulose, hydroxypropylmethyl cellulose, methylcellulose, and sodium carboxymethyl cellulose.

Active agents suitable for the present disclosure include, but are not limited to, the biologically active substances disclosed in WO 2020/251545 A1, incorporated by reference herein. Exemplary active agents include, but are not limited to, proteins (including, e.g., nucleoproteins, glycoproteins, and lipoproteins), peptides, polysaccharides and other carbohydrates, lipids, nutraceuticals, small molecules, vitamins, steroids, enzymes, nucleotides (e.g., DNA and RNA, including cDNA, shRNA, siRNA, mRNA, and modRNA), receptors, neurotransmitters, hormones, cytokines, cell adhesion sequences (e.g., RGD sequence and integrins), extracellular matrix components, growth factors, chemotactic factors, antibodies, vaccines, haptens, interferons, ribozymes, antisense agents, plasmids, aptamers, modified and naturally occurring bacteria, and viruses. Any active agent having therapeutic effects that is suitable for administration by uptake in the GI tract may be used in the compositions herein. The active agent may comprise, for example, an anti-inflammatory agent, an analgesic (e.g., acetaminophen), an antimicrobial agent, a steroid, a stem cell activating molecule, or a regenerative therapeutic agent. In at least one example, the active agent comprises a small molecule, a peptide, a protein, a nucleic acid, or an antibody.

The active agent may be suitable for treating one or more diseases or other heath conditions such as diabetes, obesity, Crohn’s disease, nonalcoholic steatohepatitis (NASH), nonalcoholic fatty liver disease (NAFLD), or cancer. In at least one example, the active agent comprises a peptide such as glucagon-like peptide 1 (GLP-1). In at least one example, the active agent comprises insulin. In at least one example, the active agent comprises metformin.

As mentioned above, the compositions herein may comprise at least one discrete layer or region of a mucoadhesive agent and at least one discrete layer or region of an active agent. The composition may be formulated so that, upon release in the GI tract, the mucoadhesive component forms at least a partial or a complete layer or coating on and around the active agent, the mucoadhesive component also forming a barrier layer with mucosa of the GI tract.

The compositions may be formulated to release in a desired region of the GI tract. Upon administration of the composition, the composition may release within a subject’s GI tract to allow the mucoadhesive component to attach to mucosal tissue, regardless of the mucosal tissue’s state e.g., healthy mucosal tissue or mucosal tissue that is diseased or otherwise compromised due to an ailment, such as an ulcer. For example, the mucosal tissue to be treated may be acidic due to an adverse health condition of the subject. Exemplary target sites for the compositions herein include the stomach, small intestine (duodenum, jejunum, and/or ileum), and large intestine (e.g., cecum and/or colon). When the composition contacts the mucosal tissue, the composition may attach to the tissue such that the active agent is in contact with, adjacent, or otherwise in immediate proximate vicinity of the tissue to allow the active agent to release from the composition, e.g., diffuse, in an inward direction toward the tissue for absorption. The mucoadhesive component of the composition may form a barrier around the active agent that prevents or otherwise inhibits the active agent from being released or diffusing toward the GI lumen. For example, when the mucoadhesive agent becomes hydrated via bodily liquid, the mucoadhesive agent may form a paste. The paste may cover the active agent as the paste attaches to the mucosa. By constraining diffusion of the active agent in a targeted direction, towards the tissue, the compositions herein may facilitate absorption of the active agent.

The compositions herein may be formulated to release the active agent within a desired period of time. For example, the composition may be formulated to release the active agent in 10 hours or less, such as 5 hours or less, 2 hours or less, or 1 hour or less. In some examples, the active agent may remain as a single phase, e.g., intact without breaking down into multiple pieces or particles. The composition may be formulated for release in a desired region of the gastrointestinal tract. For example, the composition may comprise an enteric coating, e.g., comprising one or more polymers providing for delayed release of the active agent. For example, the composition may be formulated to release the active agent in the intestine, such as the duodenum, jejunum, ileum, or the colon. In this way, the composition may be formulated for extended release in different regions of the GI tract, facilitating both local and systemic absorption. In at least one example, the composition comprises a mucoadhesive agent and an active agent; wherein the composition is formulated for release of the active agent in a first direction while inhibiting release of the active agent in a second direction opposite the first direction; wherein the composition is formulated for administration to a subject for release in a gastrointestinal tract of the subject; and wherein the composition contains an agent that hydrates to form a transient mucoadhesive paste at any pH. The viscosity of the paste may range from about 0.5-5 Pa.s at about 25° C., for example, or 0.1-50 Pa.s or higher at about 25° C.

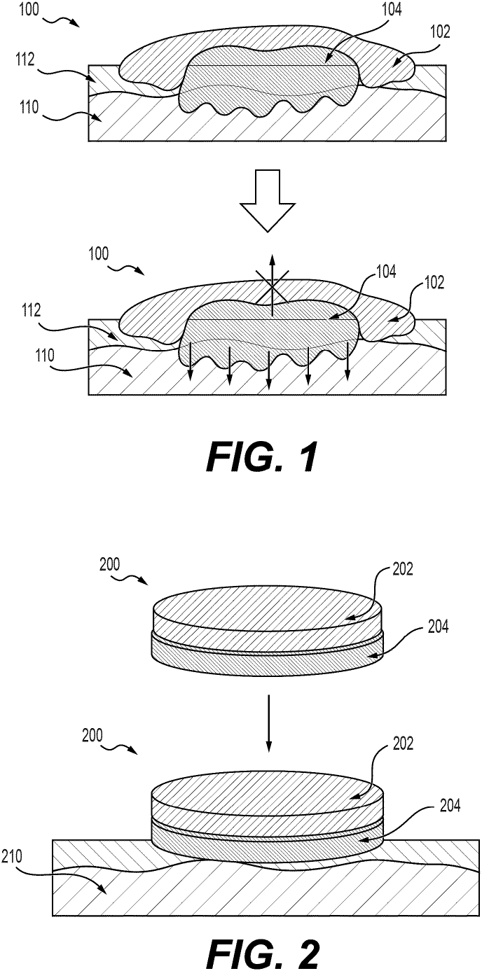

FIG. 1 depicts an exemplary composition 100 according to some aspects of the present disclosure once the composition 100 is released and attaches to mucosal tissue 110, e.g., mucosal tissue 110 including a mucus component 112. As shown, the composition 100 comprises a mucoadhesive component 102 (or mucoadhesive agent 102) and an active agent 104. In some examples, mucoadhesive component 102 may comprise acid -pretreated sucralfate, e.g., luminal coating of the intestine (“LuCI”) and active agent 104 may be referred to as a drug depot. The terms “LuCl” and “drug depot” are exemplary only and non-limiting of the types of mucoadhesive materials and active agents that may be used. Mucosal tissue may comprise healthy mucosa (e.g., healthy mucosal tissue of the stomach, duodenum, intestine, etc.), however, the same type of composition may be used with other types of mucosal tissue. The mucoadhesive component 102 (e.g., acid pretreated sucralfate or other aluminum cross-linked sulfated agent) forms a coating around the active agent 104 to establish a barrier on top of the active agent 104 (e.g., drug depot). As shown in FIG. 1, the active agent 104 directly contacts the mucosal tissue 110. As mentioned above, in some examples, mucosal tissue 110 is healthy mucosal tissue. Exemplary mucosal tissue 110 may include tissue(s) of the stomach, duodenum, or other parts of the intestine. These types of mucosal tissues are exemplary only and non-limiting of the types of mucosal tissue 110 for which the composition 100 may be used.

The lower portion of FIG. 1 illustrates the direction of the active agent 104 release into the mucosal tissue 110 and the barrier formed by the mucoadhesive agent 102 inhibiting release of the active agent 104 in the opposite direction.

The compositions herein may be in the form of a tablet or capsule, including tablets and capsules formulated as single unit compositions and multiple unit compositions providing the desired dose. For example, the composition may be formulated as a single tablet or capsule providing the desired dose of active agent. Alternatively, the composition may be formulated as multiple tablets or capsules (including, e.g., mini-tablets or mini-capsules) that collectively provide the desired dose when administered to a subject. Optionally, the composition may be formulated for delayed release, such as with an enteric coating (e.g., a coating comprising an acrylate polymer/copolymer or other sustained release polymer/copolymer).

The compositions herein may be formulated with layers. The composition may have at least two layers, e.g., at least one layer of the mucoadhesive agent and at least one layer of the active agent or a combination of active agents. The layers may have an axial or radial configuration. For example, compositions of the present disclosure may be axially layered comprising two or more layers adjacent to each other along an axis. In a radial configuration, the composition may comprise at least one layer radially outward from another layer. For example, for a cylindrical or spherical composition, a first layer and a second layer may share a common center in a cross-sectional view of the composition. The innermost layer may form a core that is covered partially or completely by a second layer. For example, the innermost layer or core may comprise and active agent and the outer layer may comprise a mucoadhesive agent. Such compositions may comprise additional layers partially or completely surrounding the outer layer and the inner layer/core, e.g., the additional layer(s) comprising one or more active agents, mucoadhesive agents, polymers or copolymers (e.g., a spacer as described below), or anti-adhesive materials.

In some examples herein, the composition includes a spacer between the mucoadhesive agent and the active agent. For example, the spacer layer may comprise one or more polymers or copolymers. The polymer/copolymer(s) may be useful as a spacer to provide separation between the mucoadhesive agent and the active agent(s), e.g., to avoid adverse reaction or interaction. The spacer may comprise one or more materials that are pharmaceutically acceptable and that do not interfere with attachment of the mucoadhesive agent to mucosal tissue or with therapeutic activity of the active agent. This separation may be provided by a layer, for example, or by combining the active agent with the spacer material(s) to limit interaction with the mucoadhesive agent. Exemplary materials useful as spacers may also serve as binders, permeation enhancers, and/or disintegrants. Exemplary materials useful as a spacer include, but are not limited to, hydroxypropylcellulose (HPC), microcrystalline cellulose (MCC), polyvinylpyrrodlidone (PVP), hydroxypropylmethyl cellulose (HPMC), lactose (monohydrate), lactose (anhydrous), poly-lactide-co-glycolic acid (PLGA), polyanhydrides, poly capro-lactone (PCL), poly vinyl acetate phthalate (PVAP), poloxomers, crospovidone and co-excipients. Exemplary co-excipients may include, but are not limited to, MCC-lactose, MCC-mannitol, MCC-guar gum, MCC-sodium carboxylmethyl cellulose (NaCMC), lactose-crospovidone, lubricants/glidants (e.g., fumed silica such as Aerosil®, magnesium stearates, sodium stearyl fumarates, talc). Incorporating a polymer/copolymer as a spacer may avoid displacement of the active agent by the mucoadhesive agent (e.g., the mucoadhesive agent occupying the interface between the composition and the tissue, inhibiting contact of the active agent(s) with the tissue. Spacers may also act as controlled release matrices for the release of active agents.

In some examples, the spacer may comprise a surfactant, e.g., to facilitate permeation of the active agent across the epithelium. Exemplary surfactants useful with spacers may include, but are not limited to, hydrophobic and/or hydrophilic based surfactants, such as sodium laureth sulfate, polyethylene glycols, and sodium N-(8-[2-hydroxybenzoyl] amino) caprylate (SNAC). Other exemplary surfactants and/or permeation enhancers may include, but are not limited to, polysorbate 80, sodium decyl sulfate (SDS), sodium lauryl sulfate (SLS), sodium octyl sulfate (SOS), N-Lauryl sarcosinate (NLS), cetyltrimethyl ammonium bromide (CTAB), benzyldimethyl dodecyl ammonium chloride (BDAC), dodecyl pyridinium chloride (DPC), palmityldimethyl ammonio propane sulfonate (PPS), chembetaine CAS (CBC), palmitoyl carnitine chloride (PCC), polyoxyethylene sorbitan monolaurate (T20), polyoxyethylene sorbitan monopalmitate (T40), sorbitan monooleate (SP80), cholic acid, hexanoic acid, isopropyl palmitate, sodium oleate, urea, lauryl amine, phenyl piperazine, sodium salicylate, and trimethoxy propylene methyl benzene.

Exemplary compositions are described below with reference to FIGS. 2-12.

FIG. 2 illustrates an exemplary layered composition 200, e.g., in tablet form that includes two layers. A first layer 202 may comprise a mucoadhesive agent (e.g., a LuCl barrier layer). The first layer 202 may also be referred to as mucoadhesive layer 202. A second layer 204 may comprise an active agent. The second layer 204 may also be referred to as active agent layer 204. As depicted in the lower portion of FIG. 2, the active agent layer 204 is available to contact mucosal tissue 210 upon administration to a subject. The mucoadhesive layer 202 may form a paste upon contact with bodily liquids and form a paste covering the active agent layer 204 to inhibit diffusion of the active agent through the composition 200 and into the GI lumen.

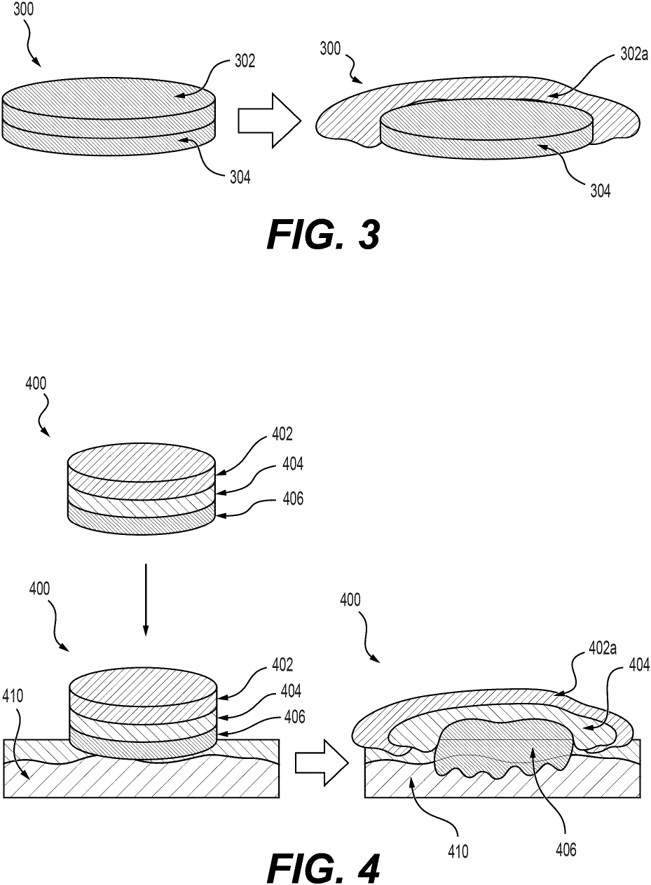

FIG. 3 illustrates another exemplary composition 300 according to the present disclosure. In FIG. 3, the composition 300 includes a first layer 302 and a second layer 304. The first layer 302 may include a mucoadhesive agent. As such, the first layer 302 may also be referred to as mucoadhesive layer 302. The second layer 304 may include a mixture of active agent and a polymer such as microcrystalline cellulose (MCC). The second layer 304 may also be referred to as active agent layer 304. Optionally, the second layer 304 may further comprise a dye (e.g., to enhance image resolution, as a marker for tissue biopsy, etc.). For example, a dye may be useful for visual observation of uptake into tissue, e.g., as part of in vitro studies. Similar to the description above referring to FIG. 2, the mucoadhesive layer 302 of FIG. 3 may form a paste 302a upon contact with bodily liquids. The paste 302a formed upon contact with bodily fluids may cover the active agent layer 304 to inhibit diffusion of the active agent through the composition 300 and into the GI lumen. In this case, mixing the active agent with the MCC (and/or other polymer(s)/copolymer(s)) in the second layer 304 may delay or modulate release of the active agent from the composition 300, provide a spacer as discussed above, and/or provide desired properties such as disintegration, hardness, and/or friability.

FIG. 4 illustrates another exemplary composition 400. In FIG. 4, the composition 400 has a layered configuration, including three layers. A first layer 402 may comprise a mucoadhesive agent (e.g., a LuCl barrier layer or layer of other aluminum cross-linked sulfated agent). As such, the first layer 402 may also be referred to as mucoadhesive layer 402. A second layer 404 is adjacent to the first layer 402. The second layer 404 may comprise a polymer or copolymer. The second layer 404 may also be referred to as spacer layer 404. A third layer 406 on the other side of the second layer 404 may comprise an active agent. The third layer 406 may also be referred to as active agent layer 406.

As shown in the lower portion of FIG. 4, the active agent layer 406 is available to contact mucosal tissue 410 upon administration to a subject. The mucoadhesive layer 402 may form a paste 402a upon contact with bodily liquids. The paste 402a formed upon contact with bodily fluids may cover the spacer layer 404 and the active agent layer 406 to inhibit diffusion of the active agent through the composition 400 and into the GI lumen. The spacer layer 404 may separate the active agent layer 406 from the paste 402a formed by the mucoadhesive layer 402.

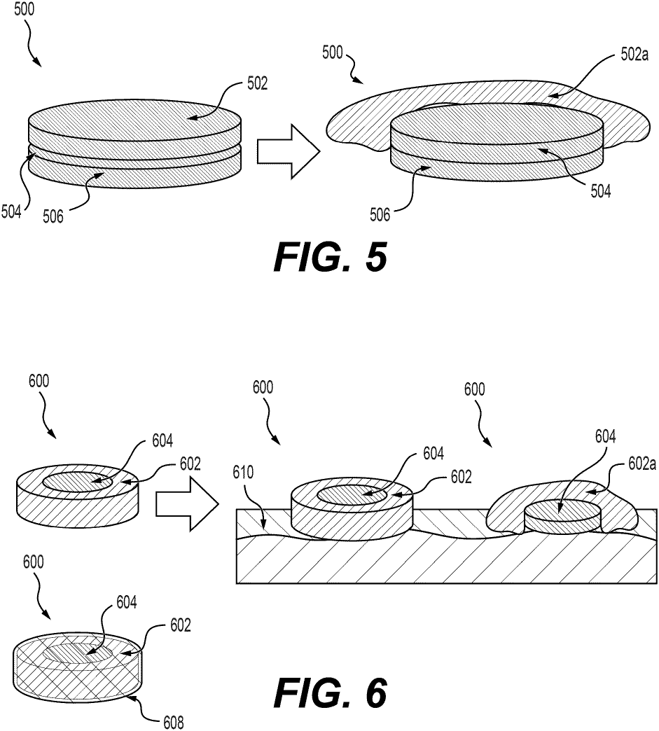

FIG. 5 illustrates an exemplary composition 500 similar to that shown in FIG. 4 (e.g., a tri-layered tablet). A first layer 502 may comprise a mucoadhesive agent. As such, the first layer 502 may also be referred to as mucoadhesive layer 502. A second layer 504 is adjacent to the first layer 502. The second layer 504 may comprise a polymer or copolymer. The second layer 504 may also be referred to as spacer layer 504. A third layer 506 on the other side of the second layer 504 may comprise an active agent. The third layer 506 may also be referred to as active agent layer 506.

FIG. 5 further depicts formation of a paste 502a, e.g., mucoadhesive paste, by the mucoadhesive agent of layer 502 in the presence of bodily liquids. The paste 502a covers both the spacer layer 504 and the active agent layer 506 to constrain the active agent layer 506 against the tissue while avoiding contact of the active agent with the mucoadhesive agent. In some examples, the spacer layer 504 may comprise MCC. In other examples, any other suitable polymer/copolymer materials may be used in the spacer layer 504. Optionally, the third (active agent) layer 506 may include a dye.

As mentioned above, the compositions herein may comprise a material or mixture of materials to inhibit moisture uptake. For example, the composition may comprise one or more anti-adhesive agents to inhibit or delay hydration of underlying layers, including a layer that includes a mucoadhesive agent or active agent. In this way, the anti-adhesive agent (or anti-adhesive layer or coating) may serve as a temporary coating for the composition to delay water uptake and premature release of the mucoadhesive agent(s) and/or active agent(s). The anti-adhesive agent(s) may resist dissolution in aqueous media for a certain period time. For example, the layer or coating of anti-adhesive agent may dissolve in aqueous media at a pH of about 1.2 to about 7.5 after approximately 5 to 10 minutes. Exemplary materials useful as anti-adhesive agents include, but are not limited to, magnesium stearate, magnesium silicate, calcium stearate, sodium lauryl sulfate, sodium stearyl fumarate, magnesium lauryl sulfate, stearic acid, calcium stearate, glyceryl behenate, behenoyl polyoxylglycerides, glyceryl dibehenate, lauric acid, glyceryl monostearate, glyceryl tristearate, myristic acid, palmitic acid, poloxamer, polyethylene glycol, polyethylene glycol 3350, polysorbate 20, polyoxyl 10 oleyl ether, polyoxyl 15 hydroxystearate, polysorbate 40, polyoxyl 20 cetostearyl ether, polyoxyl 40 stearate, polysorbate 60, polysorbate 80, potassium benzoate, sodium benzoate, sorbitan monolaurate, sorbitan monooleate, sodium stearate, sorbitan monopalmitate, sorbitan monostearate, zinc stearate, sorbitan sesquioleate, sorbitan trioleate, talc, hyaluronic acid, carboxymethylcellulose, xanthan gum, gellan gum, 2-methacryloyloxyethyl phosphorylcholine (MPC), poly(2-methacryloyloxyethyl phosphorylcholine-co-nbutyl methacrylate-co-p-vinylphenylboronic acid) (PMBV), poly(vinyl alcohol) (PVA), poly(MPC-co-methacrylic acid) (PMA), poly(MPC-co-n-butyl methacrylate) (PMB), poly(MPC-co-n-butyl methacrylate) (PMB), titanium oxide, starch, and cellulose.

FIG. 6 illustrates another exemplary composition 600 according to the present disclosure. In FIG. 6, the composition 600 includes two layers arranged radially to each other. The inner layer 604 may comprise an active agent (e.g., the active agent having therapeutic properties to provide a therapeutic layer). As such, the inner layer 604 may also be referred to as the active agent layer 604. The outer layer 602 may comprise a mucoadhesive agent (e.g., a LuCl barrier layer or other mucoadhesive agent). As such, the outer layer 602 may also be referred to as the mucoadhesive layer 602. The inner layer 604 comprising the active agent forms a core of the composition 600 and the outer layer 602 comprising the mucoadhesive agent surrounds the inner core/layer 604 of active agent. The cylindrical configuration shown in FIG. 6 allows for exposure of upper and lower surfaces of the inner active agent core/layer 604 and the outer layer 602 comprising the mucoadhesive agent to physiological media and mucosal tissue 610 upon administration to the GI system of a subject. At the same time, part of the inner active agent core/layer 604 is covered by the outer, mucoadhesive layer 602.

The mucoadhesive layer 602 may form a paste 602a upon contact with bodily liquids and cover the active agent layer 604 to inhibit diffusion of the active agent through the composition 600 and into the GI lumen. The paste 602a formed from the hydrated mucoadhesive layer 602 may attach to the mucosal tissue 610. The radially layered composition 600 may provide direct contact between the active agent layer 604 and mucosal tissue 610 while covering the remainder of the active agent layer 604 with the paste 602a formed by the mucoadhesive layer 602 to inhibit or prevent loss of active agent to the GI lumen.

Also depicted in the lower portion of FIG. 6 is an optional anti-adhesive layer or coating 608 to inhibit premature hydration of the composition 600 (e.g., water uptake by the mucoadhesive agent to form a paste) before target tissue, such as mucosal tissue 610, is reached. The anti-adhesive agent(s) within coating 608 may dissolve over time to expose the layers of mucoadhesive agent and active agent once the composition 600 reaches the mucosal tissue intended for treatment.

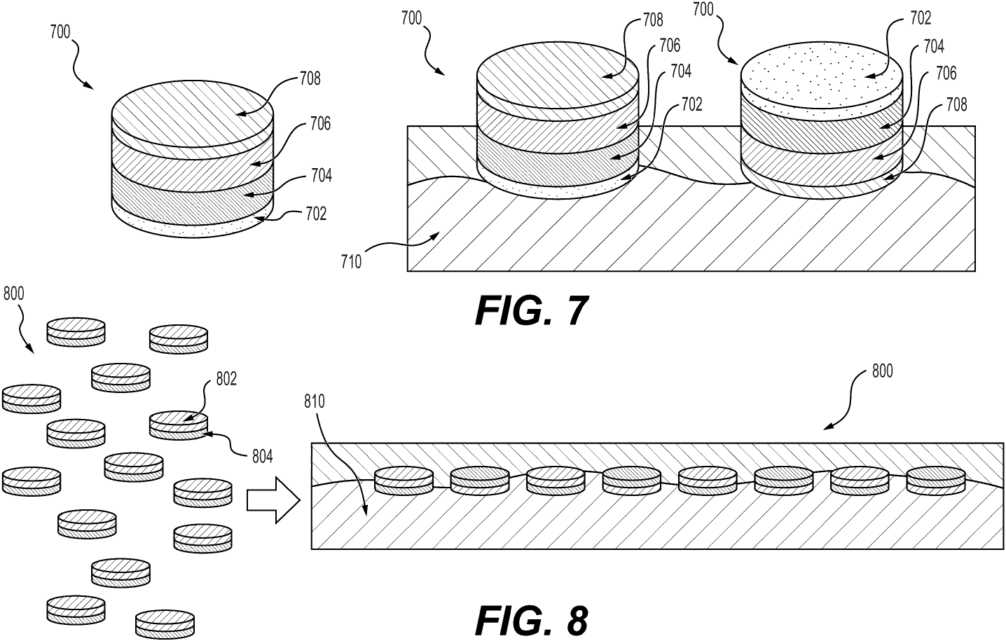

FIG. 7 illustrates another exemplary composition 700 according to the present disclosure. Similar to the composition depicted in FIG. 6 and discussed above, composition 700 includes an anti-adhesive agent formulated as an outer layer. In FIG. 7, a first layer (e.g., first outer layer) 708 may comprise an anti-adhesive agent. The first layer 708 may serve as a temporary coating that dissolves over a period of time upon contact with aqueous media. The first layer 708 may also be referred to as anti-adhesive layer 708. A second layer 706 adjacent to the first layer 708 may comprise a mucoadhesive agent (e.g., a LuCl barrier layer). The second layer 706 may also be referred to as a first mucoadhesive layer 706. A third layer 704 on the other side of the second layer 706 may comprise an active agent (e.g., therapeutic layer). The third layer 704 may also be referred to as active agent layer 704 or therapeutic layer 704. A fourth layer (e.g., second outer layer) 702 may comprise a mucoadhesive agent that may be the same or different than the mucoadhesive agent of the second layer 706. The fourth layer 702 may also be referred to as a second mucoadhesive layer 702.

FIG. 7 depicts two different scenarios for the composition 700 contacting mucosal tissue 710. For example, in one scenario the fourth layer 702 comprising a mucoadhesive agent (second mucoadhesive layer 702) contacts mucosal tissue 710 upon administration to a subject. In this scenario, the mucoadhesive agent of the fourth layer 702 may attach to the mucosal tissue 710 to allow for delivery of the active agent in the active agent layer 704 to the mucosal tissue 710. The first layer 708 comprising the anti-adhesive agent (anti-adhesive layer 708) would be the outermost layer facing the GI lumen, and its inhibition of water uptake may prevent premature hydration of the mucoadhesive agent of the second layer 706 for a more sustained delivery of the active agent in the third layer 704 (active agent layer 704). In another scenario, the first layer 708 comprising the anti-adhesive agent is in contact with the mucosal tissue 710, which may inhibit or prevent the composition 700 from attaching to the mucosal tissue 710, e.g., until the composition 700 changes position to allow the fourth layer 702 with mucoadhesive agent (second mucoadhesive layer 702) to contact the mucosal tissue 710.

As discussed above, the compositions herein may be formulated for administration to a subject as a plurality of tablets or capsules, which may be smaller in size than compositions formulated as a single tablet or capsule with an equivalent dose. This dosage form may be helpful for delivery of tablets or capsules having different types of outer layers that could contact mucosal tissue, such as the example shown in FIG. 7. A certain percentage, statistically close to 50%, of the plurality of tablets or capsules is expected to rest against tissue with the orientation desired for attachment to the tissue (e.g., the fourth layer 702 comprising a mucoadhesive agent in contact with mucosal tissue 710, rather than the first (anti-adhesive) layer 708). Thus, some of the tablets or capsules would provide for effective delivery of active agent to the tissue, while some of the tablets or capsules would not attached to tissue and may continue through the GI lumen to pass through the subject’s GI system.

FIG. 8 illustrates an exemplary composition comprising a plurality of tablets (e.g., mini-tablets) 800. The mini-tablets 800 include similar layers as composition 200 shown in FIG. 2. For example, each of the mini-tablets 800 includes a first layer 802 comprising a mucoadhesive agent and a second layer 804 comprising an active agent. The first layer 802 may also be referred to as mucoadhesive layer 802 and the second layer 804 may also be referred to as active agent layer 804 or therapeutic layer 804. When compared to a single tablet dosage form shown in FIG. 2, the total amount of active agent in the active agent layer 804 would be lower to account for delivery of multiple mini-tablets 800 to achieve a similar dose as the one tablet of FIG. 2. The schematic in FIG. 8 illustrates successful delivery of about 50% of the mini-tablets 800 wherein the first layer 802 with mucoadhesive agent contacts and attaches to mucosal tissue 810. Assuming that all of the active agent is delivered to the mucosal tissue 810 upon attachment, this would provide about 50% bioavailability relative to the total amount of active agent in the plurality of mini-tablets 800 administered to the subject.

The size and shape of tablets or capsules formulated for delivery as a plurality of units may be selected based on the desired dose of active agent, convenience for administration to a subject, among other considerations. Exemplary mini-tablets or mini-capsules may have a diameter (or largest cross-sectional diameter, if the mini-tablets or mini-capsules are not spherical in shape) size ranging from about 10 nm to about 5 mm, such as about 0.5 mm to about 2 mm, about 1 µm to about 1000 µm, or about 10 nm to about 1000 nm. Once formulated, the plurality of tablets or capsules may be packaged into a larger-sized capsule or caplet, a sachet, or any other suitable container to facilitate administration to a patient.

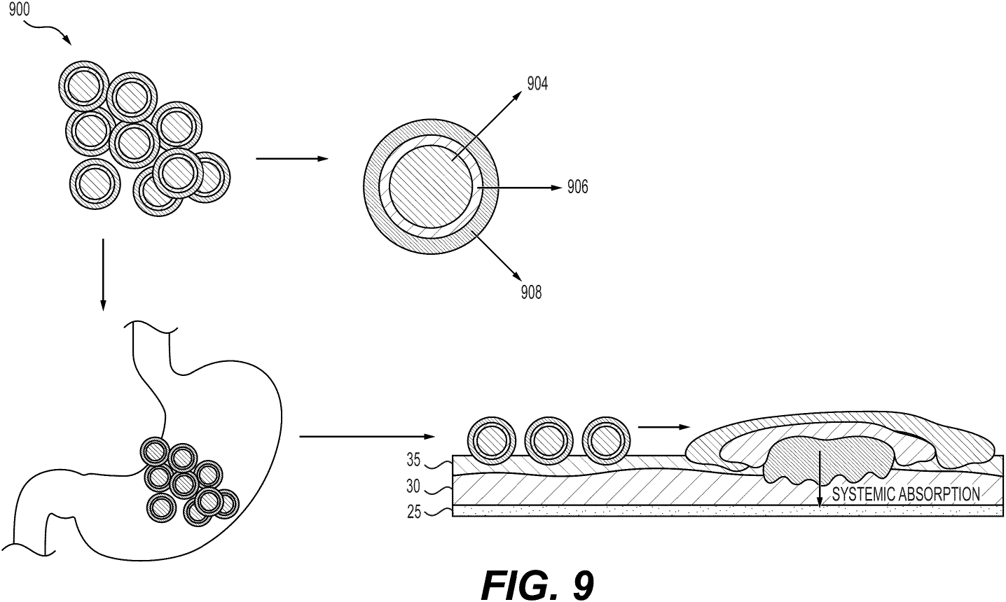

FIG. 9 illustrates another exemplary composition 900 comprising a plurality of tablets, alternatively referred to herein as granules. Each tablet may have a generally cylindrical shape that includes three layers in a radial configuration, each layer having a common center. The innermost layer or core 904 of each tablet of the composition 900 may comprise a mucoadhesive agent (e.g., a LuCl layer or other mucoadhesive agent), a second (middle) layer 906 around the core may comprise a spacer (e.g., a polymer or copolymer, disintegrant, surfactant, binder, or combination thereof), and a third, outer layer 908 may comprise an active agent (e.g., drug layer). The active agent optionally may be formulated together with a spacer material, such as a surfactant or permeation enhancer, to form the third, outer layer 908.

Once the tablets of the composition 900 shown in FIG. 9 are administered to a subject for delivery through the subject’s GI tract, a portion of each layer, including the innermost layer 904 of mucoadhesive agent, may be available for contact with mucosal tissue (the schematic in FIG. 9 showing blood circulation 25, and mucosal tissue including healthy mucosa 30 and mucus 35). Thus, assuming the largest surface area of each tablet contacts the tissue (e.g., a circular top or bottom of each cylindrical tablet), a portion of the mucoadhesive agent is available to attach to tissue, and the active agent is also in contact with tissue for delivery of the active agent. In examples where the composition also comprises a permeation enhancer (e.g., in a spacer layer or other layer of the composition), the permeation enhancer may be released upon hydration of the mucoadhesive agent, and released in combination with the active agent to facilitate uptake of the active agent and its permeation through the mucosal tissue.

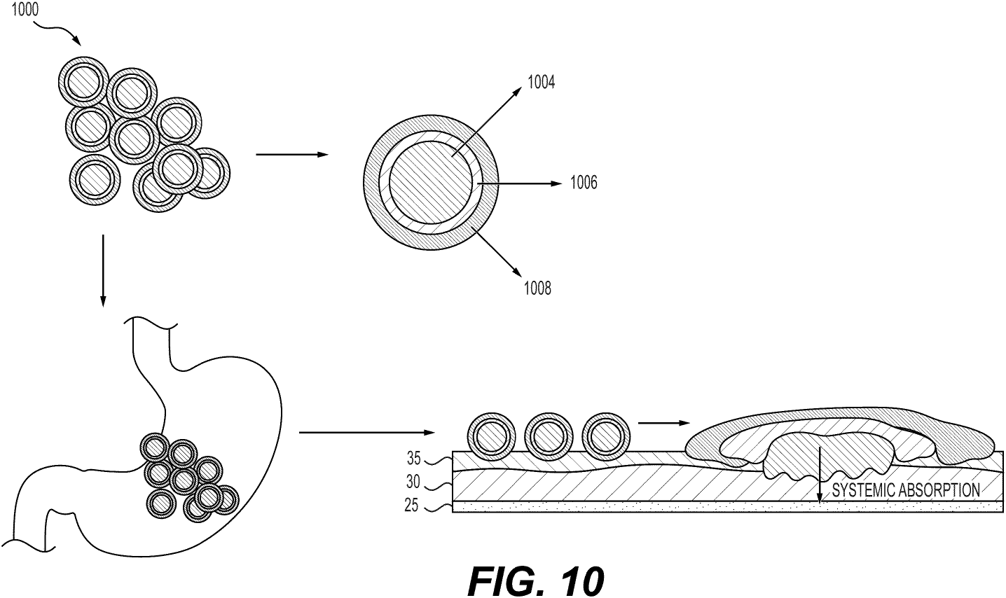

FIG. 10 illustrates another exemplary composition 1000 according to aspects of the present disclosure, formulated for administration as a plurality of tablets (or mini-tablets). Each tablet of the composition 1000 comprises three layers in a radial configuration, similar to the example shown in FIG. 9. Here, the core layer 1004 comprises an active agent (e.g., a therapeutic molecule), optionally together with a surfactant or permeation enhancer. The middle layer 1006 surrounding the core layer 1004 comprises a spacer material (e.g., a polymer or copolymer, disintegrant, surfactant, binder, or combination thereof), and the outer layer 1008 comprises a mucoadhesive agent (e.g., a LuCl layer or other mucoadhesive agent). Once the tablets of the composition 1000 shown in FIG. 10 are administered to a subject for delivery through the subject’s GI tract, a portion of each layer may be available for contact with mucosal tissue (the schematic in FIG. 10 showing blood circulation 25, and mucosal tissue including healthy mucosa 30 and mucus 35).

Upon administration to a subject, the outer layer 1008 comprising the mucoadhesive agent may be hydrated by bodily fluids to form a coating (e.g., paste) around the spacer and active agent layers 1004, 1006. Thus, the mucoadhesive agent may establish a barrier over the active agent to prevent or inhibit loss of the active agent to the GI lumen.

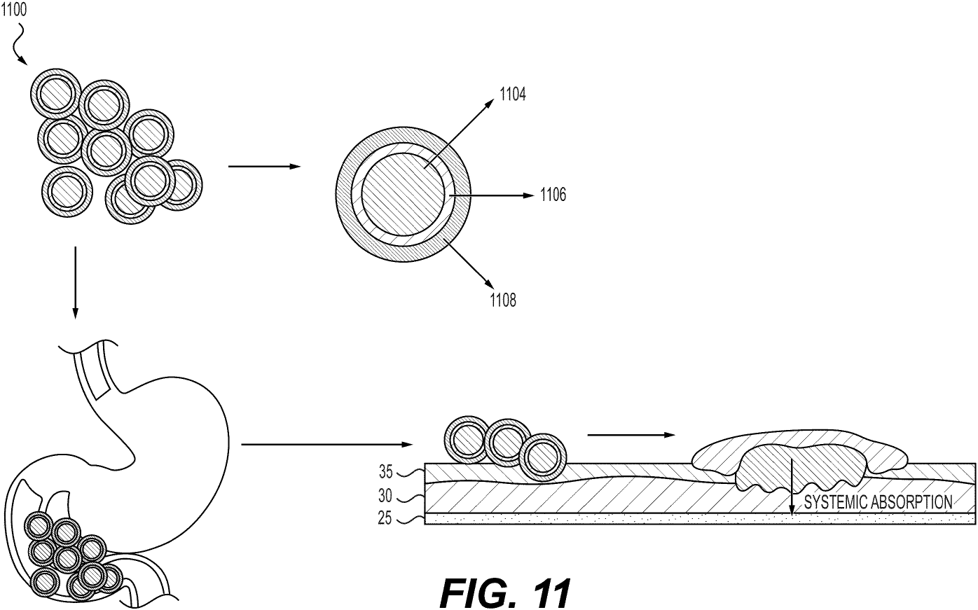

FIG. 11 illustrates another exemplary composition 1100 comprising a plurality of tablets similar to those illustrated in FIGS. 9 and 10, where the core layer 1104 comprises an active agent optionally in combination with a surfactant or permeation enhancer, the middle layer 1106 around the core comprises a mucoadhesive agent, and the outer layer 1108 comprises an enteric polymer. Exemplary enteric polymers include, but are not limited to acrylate polymers/copolymers, polymethacrylate-based copolymers, hydroxypropylmethyl cellulose (HPMC), hydroxypropyl methylcellulose acetate succinate (HPMCAS), and cellulose phthalates, among other sustained release polymers/copolymers. The outer enteric coating 1108 may delay hydration of the middle layer 1106 comprising the mucoadhesive agent and the release of the core layer 1104 comprising the active agent until reaching a target pH, such as a pH of 5 or greater. In the target pH, the outer layer 1108 may dissolve. This type of composition may be useful for administration of the active agent outside the stomach (pH generally 5 or lower), such as release in the duodenum. Once the outer layer 1108 dissolves, the middle layer 1106, comprising the mucoadhesive agent (e.g., LuCI) may be hydrolyzed when the granule is hydrolyzed upon contact with bodily fluids. The hydrolyzed mucoadhesive component then forms a coating around the spacer and the active agent (e.g., therapeutic molecule) to establish a barrier on top of the active agent (e.g., therapeutic molecule depot). The lower portion of FIG. 11 illustrates the direction of the active agent release from the therapeutic molecule depot into the mucosal tissue (the schematic in FIG. 11 showing blood circulation 25, and mucosal tissue including healthy mucosa 30 and mucus 35), wherein the mucoadhesive layer acts as a barrier against release in the opposite direction.

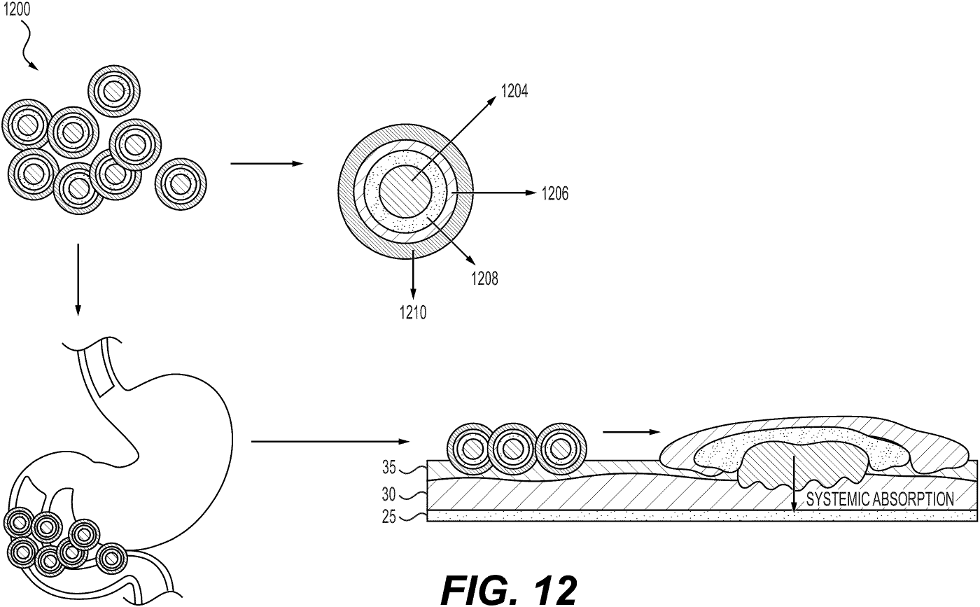

FIG. 12 illustrates another exemplary composition comprising a plurality of tablets 1200, each tablet 1200 comprising four layers in a radial configuration similar to the compositions illustrated in FIGS. 9-11 (e.g., each tablet having a generally cylindrical shape). The innermost core layer 1204 comprises an active agent optionally in combination with a surfactant or permeation enhancer, a second layer 1208 around the core layer 1204 comprises a spacer material, such as a disintegrant, surfactant, binder, or combination thereof, a third layer 1206 radially outward of the second layer 1208 comprises a mucoadhesive agent (e.g., a LuCl layer or other mucoadhesive agent), and a fourth, outermost layer 1210 comprises an enteric polymer, e.g., providing an enteric coating. For example, the enteric polymer may comprise any of the exemplary enteric polymers mentioned in connection to FIG. 11 described above. Similar to the composition 1100 of FIG. 11 described above, the enteric coating 1210 of each tablet may delay hydration of the mucoadhesive agent and active agent until a target pH, such as a pH of 5 or greater for delivery outside of the stomach. Once the tablets of the composition 1200 shown in FIG. 12 are administered to a subject for delivery through the subject’s GI tract, a portion of each layer may be available for contact with mucosal tissue (the schematic in FIG. 12 showing blood circulation 25, and mucosal tissue including healthy mucosa 30 and mucus 35).

The compositions herein may comprise about 5% by weight to about 95% by weight of a mucoadhesive agent relative to the total weight of the composition (e.g., the total weight of a capsule, tablet, minicapsule, mini-tablet, granule, etc.) such as from about 10% by weight to about 90% by weight, from about 25% by weight to about 75% by weight, from about 40% by weight to about 70% by weight, from about 50% by weight to about 90% by weight, from about 55% by weight to about 80% by weight, from about 60% by weight to about 90% by weight, from about 75% by weight to about 90% by weight, from about 55% by weight to about 70% by weight, or from about 50% by weight to about 80% by weight of a mucoadhesive agent. The mucoadhesive agent may be present in a single layer or multiple layers of the composition. In some examples, the composition comprises at least 50% by weight of a mucoadhesive agent, relative to the total weight of the composition. In some examples, the composition comprises 50% by weight or less of a mucoadhesive agent, relative to the total weight of the composition.

Additionally or alternatively, the composition may comprise about 5% by weight to about 95% by weight of one or more active agents relative to the total weight of the composition (e.g., the total weight of a capsule, tablet, minicapsule, mini-tablet, granule, etc.), such as from about 10% by weight to about 90% by weight, from about 25% by weight to about 75% by weight, from about 40% by weight to about 70% by weight, from about 50% by weight to about 90% by weight, from about 55% by weight to about 80% by weight, from about 60% by weight to about 90% by weight, from about 75% by weight to about 90% by weight, from about 55% by weight to about 70% by weight, or from about 50% by weight to about 80% by weight of one or more active agents. The active agent(s) may be present in a single layer or multiple layers of the composition. In some examples, the composition comprises at least 50% by weight active agent(s), relative to the total weight of the composition. In some examples, the composition comprises 50% by weight or less of active agent(s), relative to the total weight of the composition.

Further, for example, the compositions herein may comprise from about 0.5% by weight to about 60% by weight of one or more spacers (e.g., binder, permeation enhancer, and/or disintegrant(s)) relative to the total weight of the composition, such as from about from about 5% by weight to about 20% by weight, from about 7% by weight to about 35% by weight, from about 1% by weight to about 45% by weight, from about 3% by weight to about 15% by weight, from about 10% by weight to about 50% by weight, from about 35% by weight to about 60% by weight, from about 25% by weight to about 50% by weight, from about 45% by weight to about 55% by weight, or from about 10% by weight to about 40% by weight of spacer(s). Exemplary spacers are provided above. In some examples, the composition comprises two or more spacers in the same layer. In some examples, the composition comprises two or more layers, each or which comprises a different spacer.

An exemplary composition according to the present disclosure comprises at least 50% by weight of a mucoadhesive agent, relative to the total weight of the composition, at least one active agent, and at least one spacer. For example, the composition may comprise at least 50% by weight of a mucoadhesive agent, relative to the total weight of the composition, at least one active agent, and from about 1% by weight to about 55% by weight of one or more spacers, relative to the total weight of the composition.

The following examples are intended to illustrate the present disclosure without, however, being limiting in nature. It is understood that the present disclosure encompasses additional embodiments consistent with the foregoing description and following examples.

### EXAMPLES

The following describes experiments (Examples #1-13) performed to assess release of active agents from exemplary compositions against samples of tissue or a substrate to simulate tissue. Acetaminophen tablets were used as an exemplary active agent. Artificial stomach tissue, porcine stomach membrane, or fresh porcine stomach tissue was used as a tissue surface to investigate adhesion and release of active agents into tissue. Acid pretreated sucralfate (e.g., LuCl) was used as the exemplary mucoadhesive agent. The data herein are exemplary only and non-limiting of the compositions, methods of manufacturing, and methods of use/treatment described herein.

### Example1

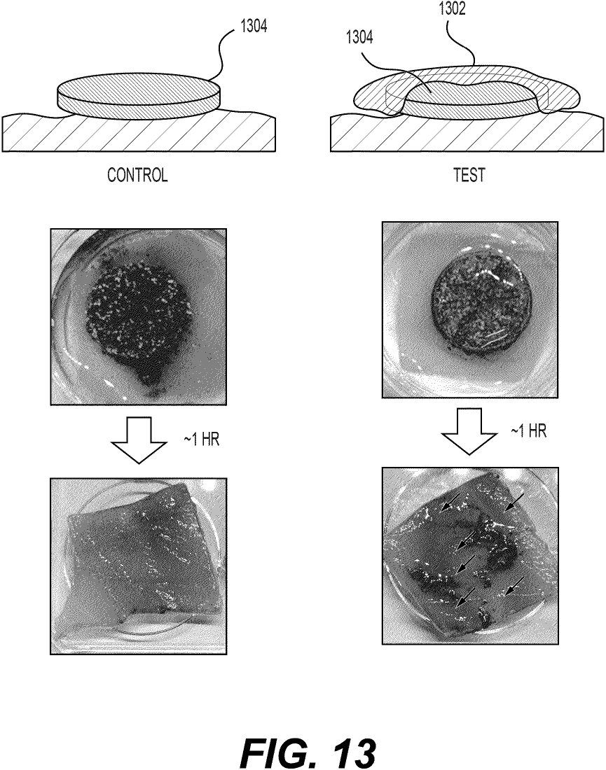

Studies were performed to evaluate release of acetaminophen tablets (Tylenol®) on artificial stomach tissue/porous membrane (SynDaver) with and without a mucoadhesive agent. The studies were performed as follows. (1) Preparation of control: a 1×1 cm² portion of artificial stomach tissue was cut and put on a well of a 6-well plate, then an acetaminophen tablet (160 mg) with dye added placed on the tissue. (2) Preparation of test sample: a 1×1 cm² portion of artificial stomach tissue was cut and put on a well of a 6-well plate; an acetaminophen tablet (160 mg) with dye added placed on the tissue; sucralfate hydrated to form a paste and about 0.3 ml of the paste (300 mg) applied to form a layer over the tablet. Illustrations in FIG. 13 show the control composition with active agent 1304 (acetaminophen tablet), and the test composition with active agent 1304 and mucoadhesive agent 1302 (acid-pretreated sucralfate). Once the control and test were set up, about 3.5-4 ml of PBS/Brij™ 35 (buffer/surfactant solution) was added so that the respective tablets were submerged. Staining was observed for about 1 hour; FIG. 13 shows photographs before and after exposure to the buffer solution/release media. The control and test compositions were observed to release most of the active agent within ~1 hr. The acid-pretreated sucralfate coated the tablet and allowed the partial disintegration of the tablet in the test composition. The mucoadhesive barrier did not interfere with contact between the active agent and the simulated tissue substrate (membrane used to simulate a tissue substrate). As the test tablet was partially disintegrated the stain from the dye observed on the membrane was less intense compared to the control (approximately 20-50%).

### Example 2

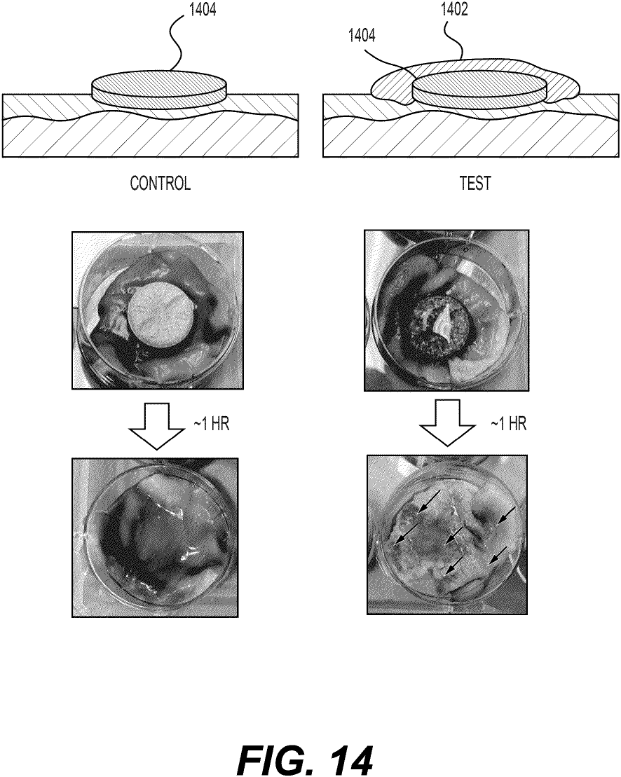

Studies were performed to evaluate release of acetaminophen tablets (Tylenol®) on porcine stomach membrane (H Mart) with and without a mucoadhesive agent. Acid pretreated sucralfate paste was prepared by adding 10 ml of PBS to 500 mg acid pretreated sucralfate. Tissue samples were prepared by cutting a small piece of stomach membrane from freshly purchased tissue and placing the stomach membrane in a 6-well plate. For the control, a tablet (160 mg) with dye added was placed on the membrane. For the test sample, a tablet (160 mg) with dye added was added to the membrane and the sucralfate paste (500 mg) added on top of the tablet. Illustrations in FIG. 14 show the control composition with active agent 1404 (acetaminophen tablet), and the test composition with active agent 1404 and mucoadhesive agent 1402 (acid-pretreated sucralfate). Next, 4 ml of PBS/Brij 35 (0.1% w/v) release media at 37° C. was added on top of both control and test samples. Samples were checked after 1 hour for blue stain on the tissue; FIG. 14 shows photographs before and after exposure to the release media. The porcine stomach membrane exhibited similar results as observed previously in Example 1 using artificial stomach membrane. The intensity of blue stain observed on the membrane for the test sample was approximately 20-50% less compared to the control. The sucralfate did not allow the tablet to disintegrate by forming a coat around it and this in turn led to the less intensity stain.

### Example 3

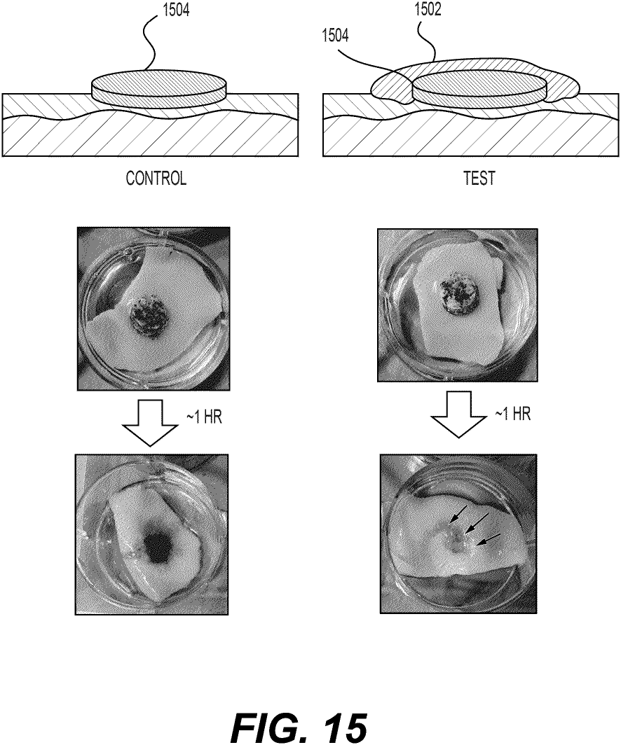

Studies were performed to evaluate release of dextran/MCC tablets on porcine stomach membrane (H Mart) with and without a mucoadhesive agent. Acid pretreated sucralfate paste was prepared as discussed in Example 2. Tissue samples were prepared by cutting a small piece of stomach membrane from freshly purchased tissue and placing the stomach membrane in a 6-well plate. Tablets were prepared by combining 100 mg of MCC powder (Roquette) with 10 mg of blue dextran powder (Sigma), blending the powders together, and forming a 100 mg tablet using a tablet punch. For the control, a tablet (100 mg) was placed on the membrane. For the test sample, a tablet (100 mg) was added to the membrane and the sucralfate paste (500 mg) added on top of the tablet. Illustrations in FIG. 15 show the control composition with active agent 1504 (MCC + dextran tablet), and the test composition with active agent 1504 and mucoadhesive agent 1502 (acid-pretreated sucralfate). Next, 4 ml of PBS/Brij 35 (0.1% w/v) release media at 37° C. was added on top of both control and test samples. Samples were checked after 1 hour for blue stain on the tissue. FIG. 15 shows photographs before and after exposure to the release media. The porcine stomach membrane exhibited similar results as observed previously in Example 1 using artificial stomach membrane. The intensity of blue stain observed on the tissue for the test sample was approximately 20-50% less compared to the control. The sucralfate did not allow the tablet to disintegrate by forming a coat around it and this in turn led to the less intensity stain.

### Example 4

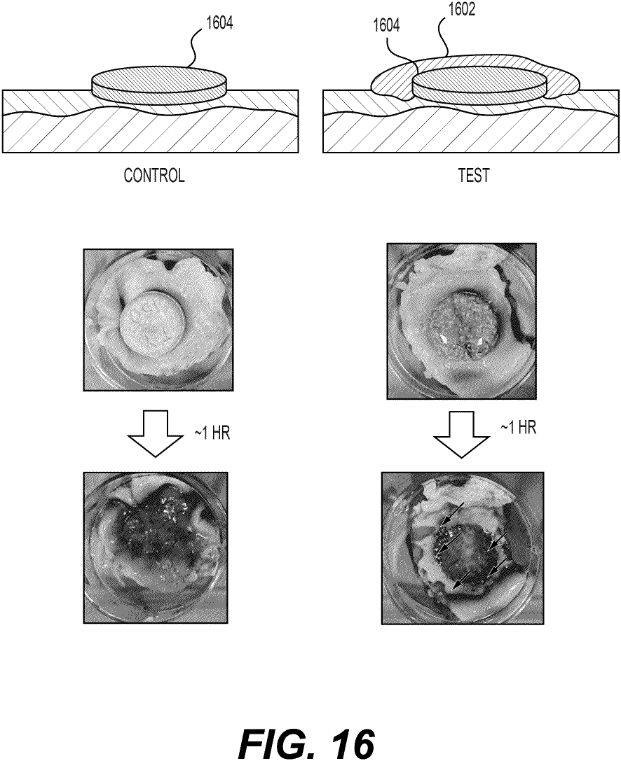

Studies were performed to evaluate release of acetaminophen tablets (Tylenol®) on fresh porcine stomach tissue with mucin (Research 87 Inc.), with and without a mucoadhesive agent. Acid pretreated sucralfate paste was prepared as discussed in Example 2. Tissue samples were prepared by cutting small pieces of stomach tissue and placing them in a 6-well plate. For the control, an acetaminophen tablet (160 mg) with dye added was placed on the membrane. For the test sample, a tablet (160 mg) with dye added was added to the membrane and the sucralfate paste (500 mg) added on top of the tablet. Illustrations in FIG. 16 show the control composition with active agent 1604 (acetaminophen tablet), and the test composition with active agent 1604 and mucoadhesive agent 1602 (acid-pretreated sucralfate). Next, 4 ml of PBS/Brij 35 (0.1% w/v) release media at 37° C. was added on top of both control and test samples. Samples were checked after 1 hour for blue stain on the tissue. FIG. 16 shows photographs before and after exposure to the release media. The intensity of blue stain observed on the tissue for the test sample was approximately 50-60% less compared to the control.

### Example 5

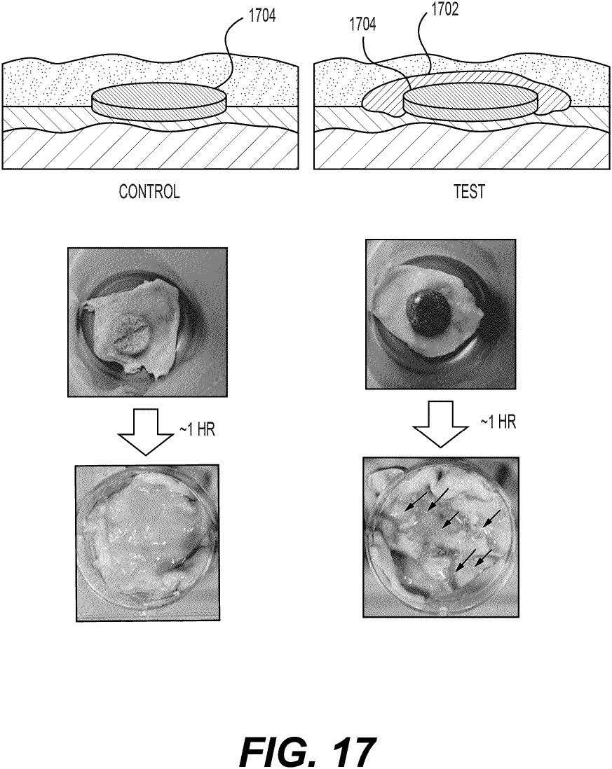

Studies were performed to evaluate release of acetaminophen tablets (Tylenol®) on fresh porcine stomach tissue with mucin (Research 87 Inc.), with and without a mucoadhesive agent in a dynamic environment (simulated with an excess of buffer). Acid pretreated sucralfate paste was prepared as discussed in Example 2. Tissue samples were prepared by cutting small pieces of the tissue and placing them in a 6-well plate. For the control, a tablet (160 mg) with dye added was placed on the tissue. For the test sample, a tablet (160 mg) with dye added was added to the membrane and the sucralfate paste (500 mg) added on top of the tablet. Illustrations in FIG. 17 show the control composition with active agent 1704 (acetaminophen tablet), and the test composition with active agent 1704 and mucoadhesive agent 1702 (acid-pretreated sucralfate). Next, 50 ml of PBS/Brij 35 (0.1% w/v) release media at 37° C. was added on top of both control and test samples. Samples were checked after 1 hour for blue stain on the tissue. FIG. 17 shows photographs before and after exposure to the release media. The intensity of blue stain observed on the tissue with acid-pretreated sucralfate was approximately better as compared to the control. The acid-pretreated sucralfate allowed more contact time in the dynamic environment provided by the release media and better staining as compared to the control where the drug washed away leading to less intense stain.

### Example 6

Studies were performed to evaluate release of bilayer dextran/PVP tablets coated with sucralfate paste and trilayer dextran/PVP/acid pretreated sucralfate tablets on fresh porcine stomach tissue with mucin (Research 87 Inc.). Acid pretreated sucralfate paste was prepared as discussed in Example 2. Bilayer tablets were prepared by pressing 10 mg of blue dextran powder (Sigma) into a layer, pressing 100 mg of PVP (povidone K30) (Frontida) into a second layer, and then punching the two layers together to form the bilayer tablet. Trilayer tablets were prepared by pressing 10 mg of blue dextran powder into a layer, pressing 50 mg of PVP into a second layer, pressing 50 mg of acid pretreated sucralfate powder into a third layer, and then punching the three layers together to form the trilayer tablet (with the PVP as the middle layer).

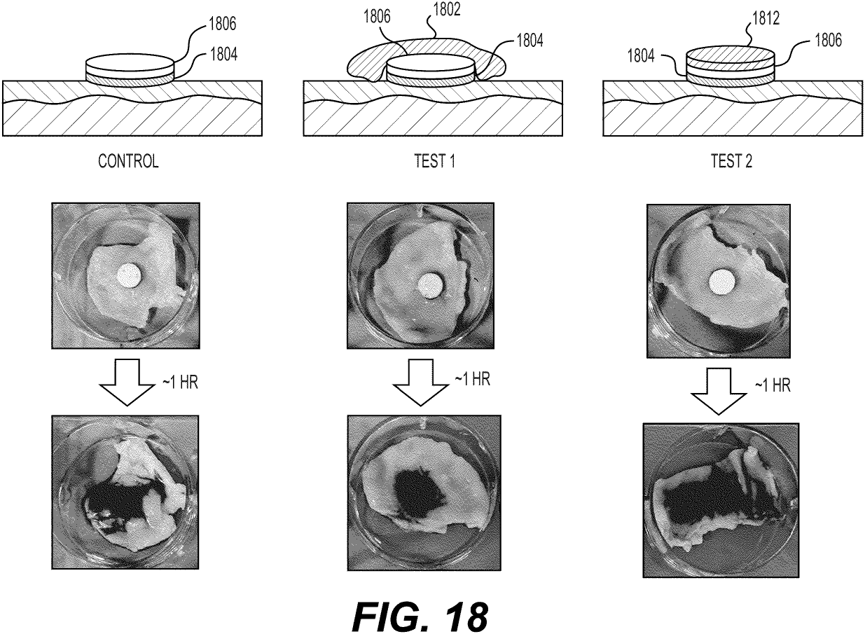

For the control, a bilayer dextran/PVP tablet was placed on a portion of tissue. For a first test sample, a bilayer dextran/PVP tablet was added to a portion of tissue and the sucralfate paste (500 mg) added on top of the tablet. For a second test sample, a trilayer dextran/PVP/sucralfate tablet was placed on a portion of tissue. Illustrations in FIG. 18 show the control composition with active agent 1804 (dextran) and spacer 1806 (PVP); test composition 1 (bilayer tablet) with active agent 1804, spacer 1806, and mucoadhesive agent as a paste 1802 (acid-pretreated sucralfate paste); and test composition 2 (trilayer tablet) with active agent 1804, spacer 1806, and mucoadhesive agent as a powder 1812 (acid pretreated sucralfate powder). Next, 4 ml of PBS/Brij 35 (0.1% w/v) release media at 37° C. was added on top of the control and test samples. Samples were checked after 1 hour for blue stain on the tissue. FIG. 18 shows photographs before and after exposure to the release media. The intensity of blue stain observed on the control, the first test sample (with sucralfate paste), and the second test sample (with sucralfate as powder in the trilayer tablet) were similar suggesting that the sucralfate does not interfere between the drug depot and tissue.

### Example 7

Studies were performed to evaluate release of bilayer and trilayer tablets on fresh porcine stomach tissue with mucin (Research 87 Inc.) in a dynamic environment. Acid pretreated sucralfate paste was prepared as discussed in Example 2. Bilayer and trilayer tablets were prepared as discussed in Example 6.

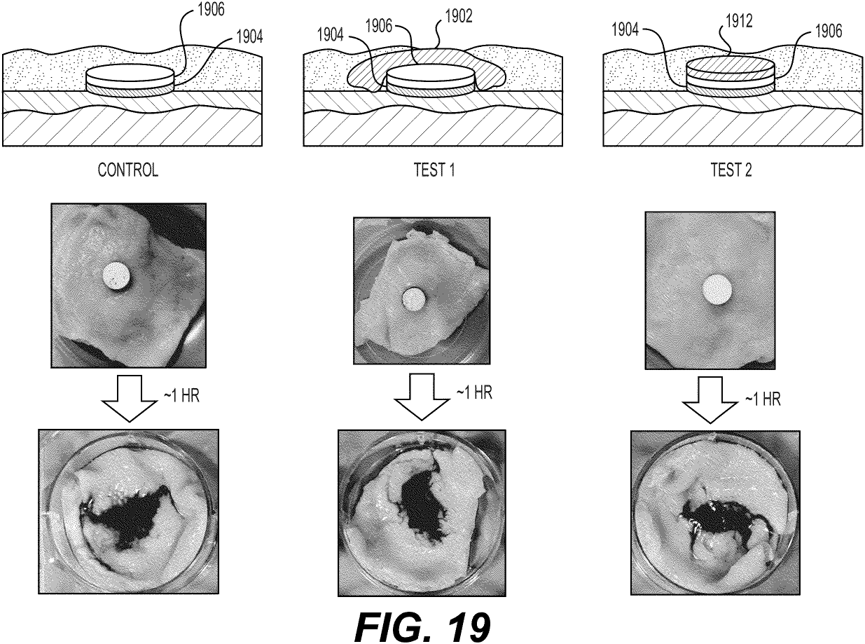

For the control, a bilayer dextran/PVP tablet was placed on a portion of tissue. For a first test sample, a bilayer dextran/PVP tablet was added to a portion of tissue and the sucralfate paste (500 mg) added on top of the tablet. For a second test sample, a trilayer dextran/PVP/sucralfate tablet was placed on a portion of tissue. Illustrations in FIG. 19 show the control composition with active agent 1904 (dextran) and spacer 1906 (PVP); test composition 1 (bilayer tablet) with active agent 1904, spacer 1906, and mucoadhesive agent as a paste 1902 (acid-pretreated sucralfate paste); and test composition 2 (trilayer tablet) with active agent 1904, spacer 1906, and mucoadhesive agent as a powder 1912 (acid pretreated sucralfate powder). Next, 50 ml of PBS/Brij 35 (0.1% w/v) release media at 37° C. was added on top of each of the control and test samples. Samples were checked after 1 hour for blue stain on the tissue. FIG. 19 shows photographs before and after exposure to the release media. The intensity of blue stain observed on the control, the first test sample (with sucralfate paste), and the second test sample (with sucralfate as powder in the trilayer tablet) were similar suggesting that the acid-pretreated sucralfate does not interfere between the drug depot and tissue even in a dynamic environment simulated by an excess of buffer.

### Example 8

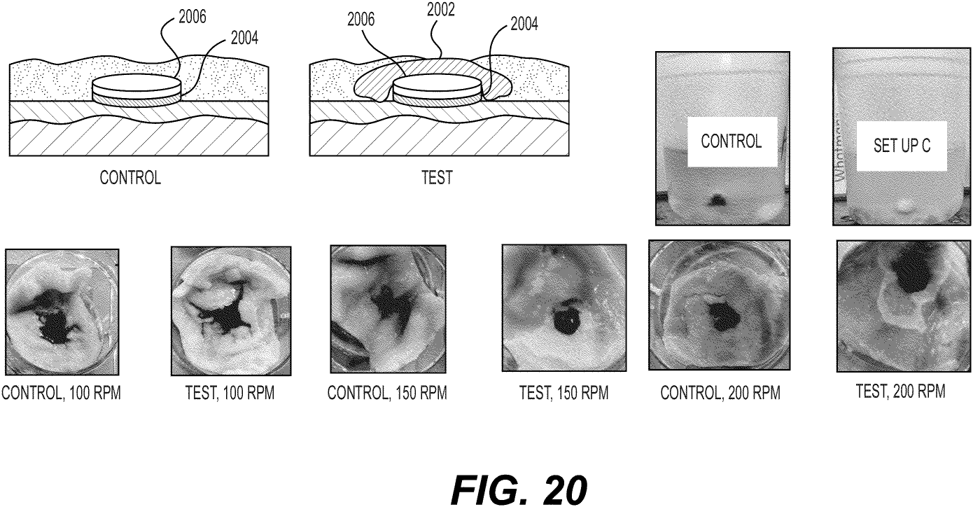

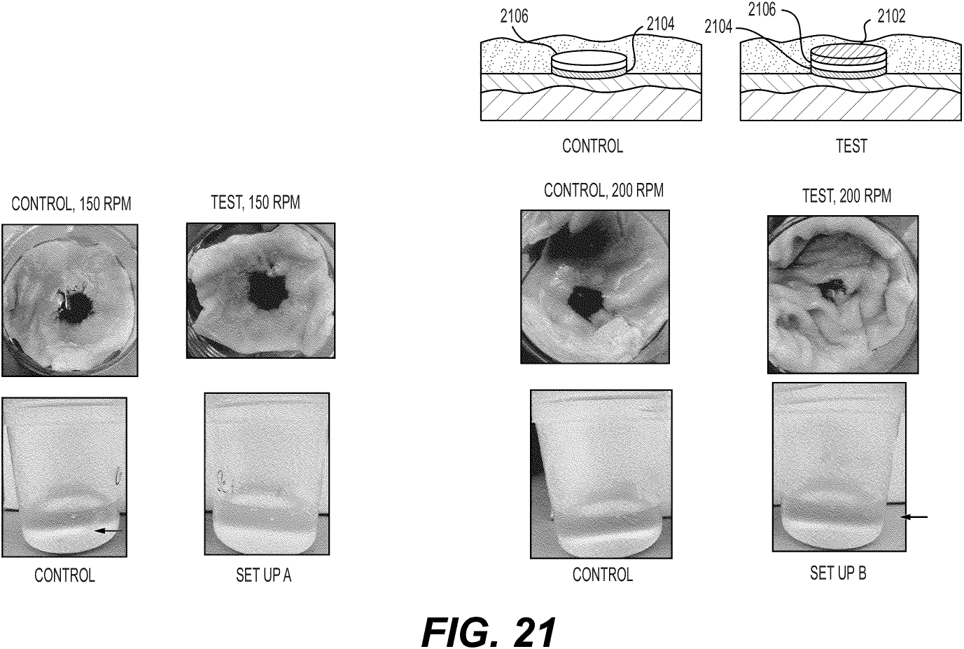

Studies were performed to evaluate release of bilayer dextran/PVP tablets on fresh porcine stomach tissue with mucin (Research 87 Inc.), with and without mucoadhesive agent in a dynamic environment with higher shear stress. Acid pretreated sucralfate paste was prepared as discussed in Example 2. Bilayer tablets were prepared as discussed in Example 6. For the control, a bilayer dextran/PVP tablet was placed on a portion of tissue. For the test sample, a bilayer dextran/PVP tablet was added to a portion of tissue and acid-pretreated sucralfate paste (500 mg) added on top of the tablet. Illustrations in FIG. 20 show the control composition with active agent 2004 (dextran) and spacer 2006 (PVP), and the test composition with active agent 2004, spacer 2006, and mucoadhesive agent 2002 (acid-pretreated sucralfate). Next, 50 ml of PBS/Brij 35 (0.1% w/v) release media at 37° C. was added on top of each sample in an incubator shaker set at 100 RPM, 150 RPM, or 200 RPM. Samples were checked after 20 minutes hour for blue stain on the tissue; see FIG. 20. The intensity of blue stain observed for the test sample as compared to the control (see upper right photographs in FIG. 20; “set-up C” refers to the incubator at 200 RPM) suggested that the sucralfate did not interfere between the drug depot and tissue, and aided the dye release better than control in the dynamic environment (excess of buffer and at higher shear stress). The absence of blue color in the buffer also suggests that the sucralfate helped to blocks the drug loss to the buffer, promoting release towards the tissue.

### Example 9

Studies were performed to evaluate release of trilayer dextran/PVP/acid pretreated sucralfate tablets on fresh porcine stomach tissue with mucin (Research 87 Inc.), in a dynamic environment with higher shear stress. Acid pretreated sucralfate paste was prepared as discussed in Example 2. Trilayer tablets were prepared as discussed in Example 6. For the control, a trilayer tablet was placed on a portion of tissue. For the test sample, a trilayer tablet was added to a portion of tissue and sucralfate paste (500 mg) added on top of the tablet. Illustrations in FIG. 21 show the control composition with active agent 2104 (dextran) and spacer 2106 (PVP), and the test composition (trilayer tablet) with active agent 2104, spacer 2106, and mucoadhesive agent in powder form 2102 (acid-pretreated sucralfate powder). Next, 50 ml of PBS/Brij 35 (0.1% w/v) release media at 37° C. was added on top of each sample in an incubator shaker set at 150 RPM (set-up A) or 200 RPM (set-up B). Samples were checked after 20 minutes hour for blue stain on the tissue; see photographs in FIG. 21. The intensity of blue stain observed for the test sample as compared to the control suggested that the sucralfate did not interfere between the drug depot and tissue, and aided the dye release better than control in the dynamic environment (excess of buffer and at higher shear stress). Blue dye present in the test tablet appeared not to have diffused to the buffer/release media, suggesting that the sucralfate prevented drug washout to the environment. The stain observed on the tissue was less intense when compared to the control at incubator setting 200 RPM.

### Example 10

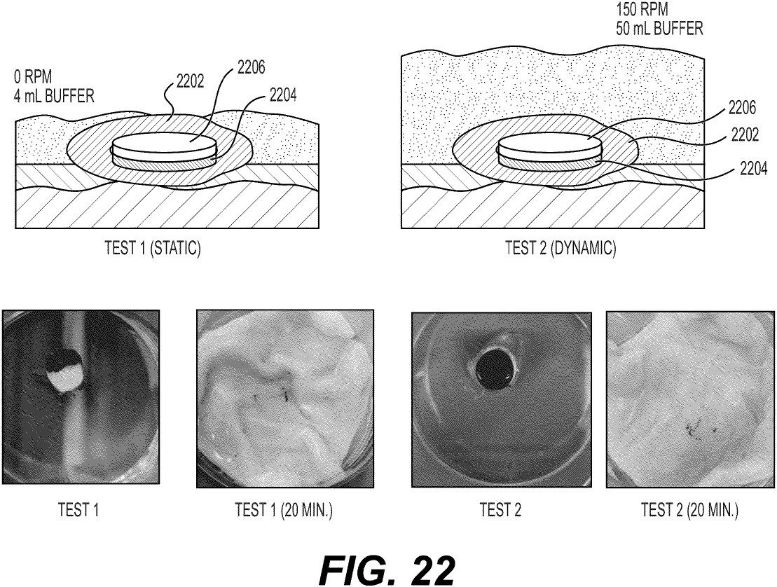

Studies were performed to evaluate release of bilayer dextran/PVP tablets on fresh porcine stomach tissue with mucin (Research 87 Inc.) in static and dynamic environments. Acid pretreated sucralfate paste was prepared as discussed in Example 2. Bilayer tablets were prepared as discussed in Example 6. Two test samples were prepared by adding sucralfate paste on portions of tissue, followed by a bilayer dextran/PVP tablet, and the tablets covered by additional sucralfate paste. In a first test sample, 4 ml of PBS/Brij 35 (0.1% w/v) release media at 37° C. was added on top of the tablet in an incubator shaker set at 0 RPM (static condition). In a second test sample, 50 ml of PBS/Brij 35 (0.1% w/v) release media at 37° C. was added on top of the tablet in an incubator shaker set at 150 RPM (dynamic condition). Illustrations in FIG. 22 show the first and second test compositions each with active agent 2204 (dextran) and spacer 2206 (PVP) surrounded by mucoadhesive agent 2202 (acid-pretreated sucralfate), exposed to static conditions and dynamic conditions. Samples were checked after 20 minutes hour for blue stain on the tissue; see photographs in FIG. 22. The intensity of blue stain observed on the tissue for both the static and dynamic conditions was less than in previous samples (without sucralfate paste between tablets and tissue). This suggests that the sucralfate interfered between the drug depot and tissue when added as a paste at the bottom and hindered the drug release towards the tissue in static and dynamic conditions.

### Example 11

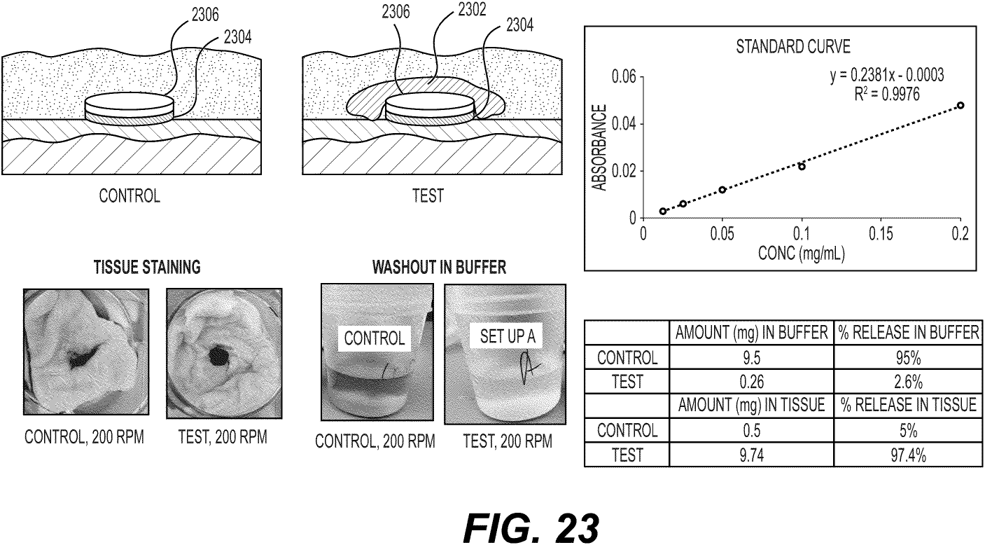

Studies were performed to evaluate the release profile of bilayer dextran/PVP tablets on fresh porcine stomach tissue with mucin (Research 87 Inc.), with and without mucoadhesive agent in a dynamic environment with higher shear stress. Acid pretreated sucralfate paste was prepared as discussed in Example 2. Bilayer tablets were prepared as discussed in Example 6. For the control, a bilayer dextran/PVP tablet was placed on a portion of tissue. For the test sample, a bilayer dextran/PVP tablet was added to a portion of tissue and sucralfate paste (500 mg) added on top of the tablet. Illustrations in FIG. 23 show the control composition with active agent 2304 (dextran) and spacer 2306 (PVP), and the test composition with active agent 2304, spacer 2306, and mucoadhesive agent 2302 (acid-pretreated sucralfate). Next, 50 ml of PBS/Brij 35 (0.1% w/v) release media at 37° C. was added on top of each sample in an incubator shaker set at 200 RPM. Samples were checked after 20 minutes for blue stain released into the buffer vs. the amount retained on the tissue; see photographs in FIG. 23.

The amount of blue dye released into the buffer media was quantified via spectrophotometry. For each sample, 300 µl of the supernatant (buffer/media) was aliquoted into a 96-well plate the absorbance at 610 nm recorded. The absorbance value was used to calculate the amount of dye released into the buffer using a standard curve prepared with serial dilutions of blue dextran dye in PBS/Brij 35. The amount of dye released into the tissue was calculated based on the total amount of dye (10 mg), subtracting the quantity released into the buffer. For the control, 9.5 mg of dye released into the buffer, leaving only 0.5 mg of dye retained on the tissue. For the test sample, 0.26 mg of dye released into the buffer, leaving 9.74 mg of dye retained on the tissue. These results show that LuCl acted as a barrier that prevented drug release into the buffer side (preventing about 92.4% of drug washout) and further provided one directional release.

### Example 12

Studies were performed to evaluate the release profile of bilayer dextran/PVP and dextran/acid pretreated sucralfate matrix tablets on fresh porcine stomach tissue with mucin (Research 87 Inc.) a dynamic environment. Bilayer dextran/PVP tablets were prepared as discussed in Example 6. A different, second bilayer tablet was prepared with acid pretreated sucralfate incorporated into a matrix formulation. The matrix formulation was prepared by combining acid pretreated sucralfate (“LuCl”) in powder form with lactose, fumed silica and PVP. This sucralfate matrix powder mixture (130.6 mg) was pressed into a layer, 10 mg of blue dextran pressed into a second layer, and the two layers punched together to form a tablet.

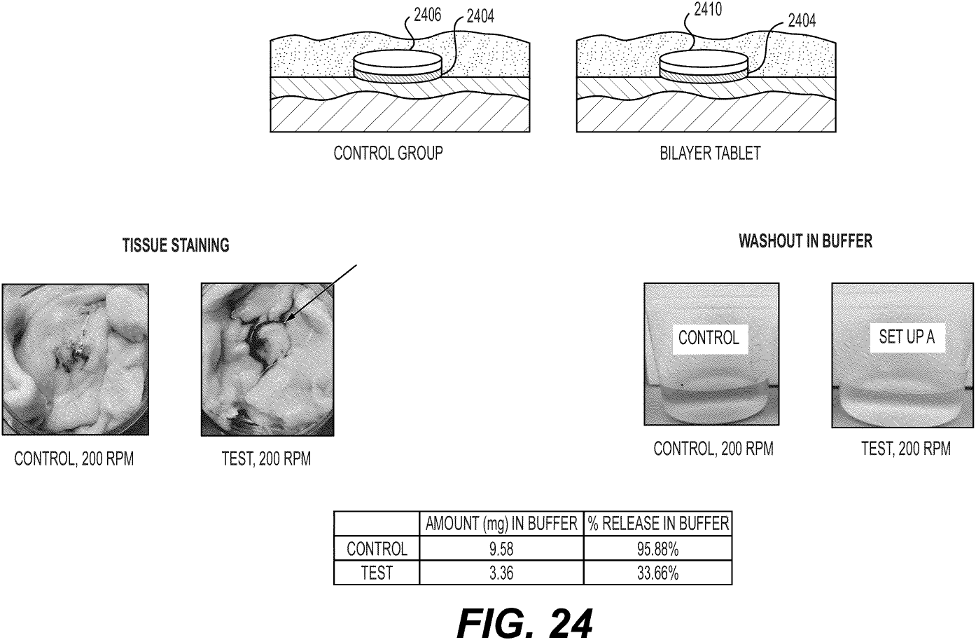

For the control, a bilayer dextran/PVP tablet was placed on a portion of tissue. For the test sample, a sucralfate matrix/dextran bilayer tablet was added to a portion of tissue. Illustrations in FIG. 24 show the control composition with active agent 2404 (dextran) and spacer 2406 (PVP), and the test composition with active agent 2404 and mucoadhesive agent matrix 2410 (acid-pretreated sucralfate/lactose/fumed silica/PVP). Next, 50 ml of PBS/Brij 35 (0.1% w/v) release media at 37° C. was added on top of each sample in an incubator shaker set at 200 RPM. Samples were checked after 20 minutes for blue stain released into the buffer vs. the amount retained on the tissue; see FIG. 24. The amount of dye released into the buffer as compared to the amount retained on tissue was calculated as discussed in Example 11. For the control, 9.58 mg was released into the buffer, indicating that only 0.42 mg of dye was retained on the tissue. For the test sample (“set-up A”), 3.36 mg was released into the buffer, indicating that 6.64 mg of dye was retained on the tissue. These results suggest that the sucralfate matrix formulation acted as a barrier to prevent drug release into the buffer side, providing one directional release.

### Example 13

Studies were performed to evaluate the release profile of bilayer dextran/PVP and trilayer dextran/PVP/acid pretreated sucralfate matrix tablets on fresh porcine stomach tissue with mucin (Research 87 Inc.) a dynamic environment. Bilayer dextran/PVP tablets were prepared as discussed in Example 6. Trilayer tablets with differing amounts of acid pretreated sucralfate were prepared by pressing a first layer of 80 mg of acid pretreated sucralfate matrix formulation, a second layer of 10 mg blue dextran powder, and a third layer of 20 mg PVP powder, and the punching the layers together to form the trilayer tablet (with PVP as the middle layer). The matrix formulations (#1, #2, #3, and #4) were prepared by combining acid pretreated sucralfate in powder form with lactose, fumed silica and PVP.

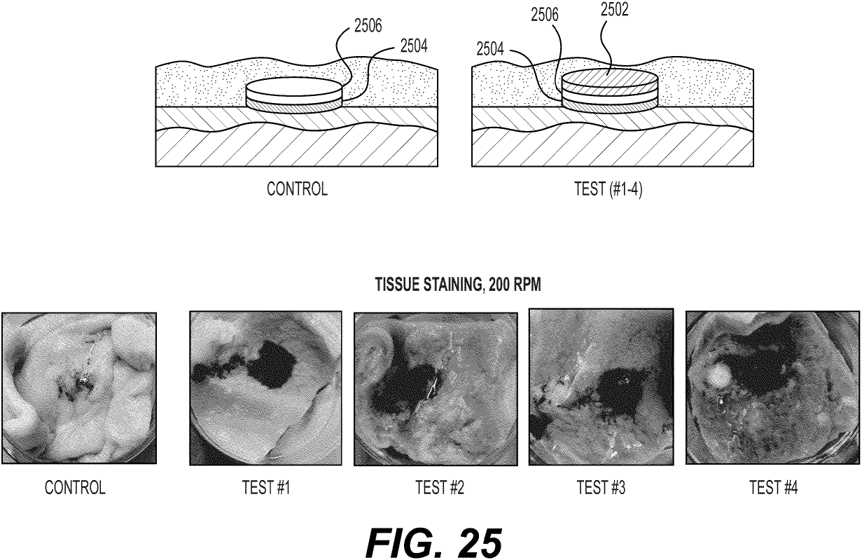

For the control, a bilayer dextran/PVP tablet was placed on a portion of tissue. For the test samples, a trilayer tablet was added to a portion of tissue. Illustrations in FIG. 25 show the control composition with active agent 2504 (dextran) and spacer 2506 (PVP), and test compositions #1-4 (trilayer tablets) with active agent 2504, spacer 2506, and mucoadhesive agent matrix 2502 (acid-pretreated sucralfate/lactose/fumed silica/PVP). Next, 50 ml of PBS/Brij 35 (0.1% w/v) release media at 37° C. was added on top of each sample in an incubator shaker set at 200 RPM. Samples were checked after 20 minutes for blue stain released into the buffer vs. the amount retained on the tissue. The amount of dye released into the buffer as compared to the amount retained on tissue was calculated as discussed in Example 11; see photographs in FIG. 25. For the control, 95.88% of the dye was released into the buffer. For the test samples #1-#4, the results ranged from about 8% to about 26% washout. These results suggest that the sucralfate matrix formulation acted as a barrier to prevent drug release into the buffer side, providing one directional release. These results indicate that the sucralfate matrix formulation acted as a barrier to prevent drug release into the buffer side and provide one directional release. Sample #4 had the greatest effect on preventing drug washout (about 92% retention on tissue).

It is intended that the specification with examples and the figures be considered as exemplary only, with a true scope and spirit of the present disclosure being indicated by the following claims.

1. A composition comprising:
a mucoadhesive agent, wherein the mucoadhesive agent comprises an aluminum crosslinked sulfated agent that forms a paste with an aqueous liquid in the absence of acid; and
an active agent;
wherein the composition is formulated for release of the active agent in a first direction while inhibiting release of the active agent in a second direction opposite the first direction; and
wherein the composition is formulated for administration to a subject for release in a gastrointestinal tract of the subject.

2. The composition of claim 1, wherein the composition is formulated as an osmotic pump, a drug delivery device, a tablet, a plurality of tablets, a capsule, or a plurality of capsules for oral administration.

3. The composition of claim 1, wherein the composition includes an enteric polymer, an anti-adhesive agent, or both.

4. The composition of claim 1, wherein the composition is in the form of a tablet that includes a first layer comprising the mucoadhesive agent and a second layer comprising the active agent.

5. The composition of claim 4, wherein the first and second layers have an axial configuration.

6. The composition of claim 4, wherein the first and second layers have a radial configuration.

7. The composition of claim 1, further comprising a spacer material between the mucoadhesive agent and the active agent, wherein the spacer material comprises a polymer, copolymer, binder, permeation enhancer, disintegrant, surfactant, or combination thereof.

8. The composition of claim 7, wherein the composition is in the form of a tablet that includes a first layer comprising the mucoadhesive agent, a second layer comprising the spacer material, and a third layer comprising the active agent.

9. The composition of claim 7, wherein the spacer material comprises a cellulose polymer, polyvinylpyrrodlidone, or a combination thereof.

10. The composition of claim 1, wherein the active agent is mixed with a polymer, copolymer, permeation enhancer, surfactant, or combination thereof.

11. The composition of claim 1, wherein the mucoadhesive agent is a first mucoadhesive agent and the composition further comprises a second mucoadhesive agent different from the first mucoadhesive agent.

12. The composition of claim 1, wherein the mucoadhesive agent comprises aluminum hydroxide crosslinked with sulfated sucrose, sulfated dextran, sulfated dextrin, sulfated amylopectin, sulfated amylose, sulfated cellulose, carrageenan, chondroitin sulfate, glucose sulfate, sucrose sulfate, heparin, heparin sulfate, or raffinose sulfate.

13. The composition of claim 1, wherein the active agent comprises a protein, peptide, carbohydrate, lipid, nutraceutical, small molecule, vitamin, steroid, enzyme, nucleotide, receptor, neurotransmitter, hormone, cytokine, cell adhesion sequence, extracellular matrix component, growth factor, chemotactic factor, antibody, vaccine, hapten, interferon, ribozyme, antisense agent, plasmid, aptamer, bacteria, virus, anti-inflammatory agent, analgesic, antimicrobial agent, steroid, stem cell activating molecule, or regenerative therapeutic agent, or a combination thereof.

14. The composition of claim 13, wherein the active agent comprises glucagon-like peptide 1 (GLP-1), insulin, or metformin.

15. A method of treating a subject, comprising administering the composition of claim 1 to the subject, wherein the composition forms a mucoadhesive coating on a stomach, esophagus, small intestine, or large intestine of the subject.

16. The method of claim 15, wherein the subject has a health condition chosen from diabetes, obesity, Crohn’s disease, nonalcoholic steatohepatitis, nonalcoholic fatty liver disease, Barrett’s esophagus, or cancer, and the active agent treats the health condition.

17. The method of claim 15, wherein the mucoadhesive coating forms on healthy tissue, or tissue that is diseased.

18. A composition comprising:
a mucoadhesive agent, wherein the mucoadhesive agent comprises an aluminum crosslinked sulfated agent that forms a paste with an aqueous liquid in the absence of acid; and
an active agent;
wherein the composition includes a first layer comprising the mucoadhesive agent and a second layer comprising the active agent, wherein the first and second layers have an axial configuration or a radial configuration; and
wherein the composition is formulated for administration to a subject for release in a gastrointestinal tract of the subject.

19. The composition of claim 18, wherein the composition further comprises a third layer comprising a spacer material, the spacer material comprising a polymer, copolymer, binder, permeation enhancer, disintegrant, surfactant, or combination thereof, wherein the third layer is between the first layer and the second layer.

20. The composition of claim 18, wherein the active agent comprises a protein, peptide, carbohydrate, lipid, nutraceutical, small molecule, vitamin, steroid, enzyme, nucleotide, receptor, neurotransmitter, hormone, cytokine, cell adhesion sequence, extracellular matrix component, growth factor, chemotactic factor, antibody, vaccine, hapten, interferon, ribozyme, antisense agent, plasmid, aptamer, bacteria, virus, anti-inflammatory agent, analgesic, antimicrobial agent, steroid, stem cell activating molecule, or regenerative therapeutic agent, or a combination thereof.
